(19) United States
(12) Patent Application Publication
(10) Pub. No.: US 2024/0216312 A1
(43) Pub. Date: Jul. 4, 2024

(54) PPAR-AGONISTS FOR USE IN THE TREATMENT OF LIVER FAILURE

(71) Applicant: Genfit, Loos (FR)

(72) Inventors: Vanessa Legry, Emmerin (FR); Remy Hanf, Lille (FR); Simon Debaecker, Pont-A-Marcq (FR); Philippe Poulain, Lorgies (FR); Benoit Noel, Gondecourt (FR)

(21) Appl. No.: 18/289,211

(22) PCT Filed: May 10, 2022
(86) PCT No.: PCT/EP2022/062707

§ 371 Date: Nov. 1, 2023

(30) Foreign Application Priority Data
May 11, 2021 (EP) 21305614.6

(51) Int. Cl. A61K 31/192; A61P 1/16

(52) U.S. Cl. CPC A61K 31/192; A61P 1/16

(57) ABSTRACT

The invention relates to compounds for use in the treatment of liver failure.

The invention is in the medical field, and relates to compounds for use in the treatment of liver failure.

### BACKGROUND OF THE INVENTION

Liver failure is a severe inability of the liver to perform its normal functions. Manifestations of liver failure herein include acute liver failure (ALF), decompensated cirrhosis, acute cirrhosis decompensation (AD), and acute on chronic liver failure (ACLF).

Acute liver failure (ALF) The term “ALF” describes a disorder characterized by an acute loss of liver function in the absence of pre-existing chronic liver disease. ALF has also been referred to as fulminant hepatic failure, acute hepatic necrosis, fulminant hepatic necrosis, and fulminant hepatitis. ALF is a rare and severe consequence of abrupt hepatocyte injury, and can evolve over days or weeks to a lethal outcome. A variety of insults to liver cells result in a consistent pattern of rapid-onset elevation of aminotransferases, altered mentation, and disturbed coagulation. The absence of existing liver disease distinguishes ALF from liver failure due to end-stage chronic liver disease (decompensated cirrhosis, acute decompensation and acute-on-chronic liver failure). In ALF, substances that lead to hepatocyte injury cause either direct toxic necrosis, or apoptosis and immune injury, which is a slower process. The time from the onset of symptoms to the onset of hepatic encephalopathy distinguishes the different forms of acute liver failure: a direct, very rapid injury (within hours), referred to as hyperacute liver failure; and a slower, immune-based injury (days to weeks), considered acute or subacute. The term “hepatic encephalopathy”, or HE, as used herein refers to the occurrence of confusion, altered level of consciousness and coma as a result of liver failure. In the advanced stages it is called hepatic coma or coma hepaticum. The five most prevalent causes of ALF in developed countries are paracetamol (acetaminophen) toxicity, ischaemia, drug-induced liver injury, hepatitis B, and autoimmunity, which account for nearly 80% of cases. Hepatitis A, B, and E are the main causes of ALF in developing countries. The remaining causes of ALF comprise fewer than 15% of the total and include heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex, and diffusely infiltrating malignancies. Untreated, the prognosis is poor, so timely recognition and management of patients with acute liver failure is crucial. Whenever possible, patients with acute liver failure should be managed in an intensive care unit at a liver transplantation center.

### Decompensated Cirrhosis and Acute Decompensation (AD)

The term “cirrhosis” as used herein, refers to a condition characterized by replacement of liver tissue by fibrosis and regenerative nodules which lead to loss of liver function up to decompensation. Ascites (fluid retention in the abdominal cavity) is the most common complication associated with cirrhosis decompensation. It is associated with a poor quality of life, increased risk of infection and poor long-term outcome. Other potentially life-threatening complications are hepatic encephalopathy and bleeding from esophageal varices. Cirrhosis decompensation has many possible clinical manifestations. These signs and symptoms may be either as a direct result of the failure of liver cells or secondary to the resultant portal hypertension. Effects of portal hypertension include splenomegaly, gastroesophageal varices, and portocollateral circulation as a result of formation of venous collateral veins between portal system and the periumbilical veins as a result of portal hypertension.

Cirrhosis is divided in two clinical categories: compensated and decompensated cirrhosis.

The term “compensated cirrhosis” as used herein, means that the liver is heavily scarred but can still perform many important bodily functions. Patients suffering from compensated cirrhosis experience few or no symptoms and can live without serious clinical complications. Patients at early stages of compensated cirrhosis are characterized by low levels of portal hypertension and lack of esophageal varices. Patients at advanced stages of compensated cirrhosis are characterized by higher levels of portal hypertension and presence of esophageal varices but without ascites and without bleeding.

The term “decompensated cirrhosis” as used herein, means that the liver is extensively scarred and unable to function properly. Patients suffering from decompensated cirrhosis develop a variety of symptoms such as fatigue, loss of appetite, jaundice, weight loss, ascites and/or edema, hepatic encephalopathy and/or bleeding. Patients at early stages of decompensated cirrhosis are characterized by the presence of ascites with or without esophageal varices in a patient that has never bled. Patients at advanced stages of decompensated cirrhosis are characterized by more sever ascites alone or in association with bleeding, bacterial infections and/or hepatic encephalopathy. Complications associated with decompensated cirrhosis such as ascites, edema, bleeding problems, bone mass and bone density loss, hepatomegaly, menstrual irregularities in women and gynecomastia in men, impaired mental status, itching, kidney function failure and muscle wasting can be developed.

The term “acute decompensation” refers to an abrupt deterioration of liver function in patients with advanced chronic liver diseases, compensated cirrhosis or stable decompensated cirrhosis requiring immediate hospitalization. At hospital admission, patients with AD have multiple symptoms including, severe ascites, hepatic encephalopathy, variceal bleeding associated or not with sepsis and/or impaired renal function and/or coagulopathy and/or impaired cardiovascular function and/or impaired respiratory function. AD is a life-threatening condition with an overall mortality rate of 11% at 28-Days.

### Acute on Chronic Liver Failure (ACLF)

ACLF is the most serious hepatic condition observed in patients with known chronic liver disease who have acute decompensation of liver function.

ACLF is an abrupt and life-threatening worsening of clinical conditions in patients with advanced cirrhosis or with cirrhosis due to a chronic liver disease. Three major features characterize this syndrome: it generally occurs in the context of intense systemic inflammation, frequently develops in close temporal relationship with proinflammatory precipitating events (e.g., infections or alcoholic hepatitis), and is associated with single- or multiple-organ failure affecting minimal functioning of vital organs: liver, kidneys, brain, coagulation and/or cardiovascular functions and/or respiratory system. As for sepsis, organ failures are identified with the use of a modified Sequential Organ Failure Assessment score (DOFA score) or the EASL-CLIF Consortium organ failure scoring system), which considers the function of the liver, kidney, and brain, as well as coagulation, circulation, and respiration, allowing stratification of patients in subgroups with different risks of death. Several classifications have been proposed for grading ACLF (APASL, EASL/CLIF, NASCELD). Using the EASL/CLIF, patients were stratified into four prognostic grades according to the number of organ failures at diagnosis (no acute-on-chronic liver failure and acute-on-chronic liver failure grades 1, 2, and 3). Predisposition to ACLF is correlated to the severity (i.e. fibrosis advancement up to cirrhosis) of underlying chronic liver disease. Irrespective of the underlying chronic liver disease, (cholestatic, metabolic liver diseases, chronic viral hepatitis and nonalcoholic steatohepatitis (NASH), alcoholic hepatitis) compensated cirrhosis and stable decompensated cirrhosis are the main conditions associated with development of ACLF. Alcoholic cirrhosis constitutes 50-70% of all underlying liver diseases of ACLF in Western countries, whereas viral hepatitis-related cirrhosis constitutes about 10-30% of all cases.

The severity of underlying disease can be assessed by the Model for End-Stage Liver Disease (MELD) scores.

ACLF requires a precipitating event that occurs in the setting of cirrhosis and/or chronic liver disease, and progresses rapidly to multiorgan failure with high mortality. The precipitating events may be reactivation of hepatitis B or superimposed viral hepatitis, alcohol, drugs, ischemic, surgery, sepsis or idiopathic. However, about 40% of patients with ACLF have no precipitating events.

At the onset of liver failure, translocation of bacterial products with or without concomitant translocation of living bacteria from the intestinal lumen plays a pivotal role in development of multiple organ dysfunctions and failures via intense systemic inflammatory response syndrome.

Host response determines the severity of injury. Inflammation and neutrophil dysfunction are of major importance in the pathogenesis of ACLF, and a prominent pro-inflammatory cytokine profile causes the transition from stable decompensated cirrhosis to AD and eventually ACLF. In these patients, an inflammatory response may lead to immune dysregulation, which may predispose to infection that would then further aggravate a pro-inflammatory response resulting in a vicious cycle. Cytokines are believed to play an important role in ACLF. Elevated serum levels of several cytokines, including tumor necrosis factor (TNF)-α, sTNF-αR1, sTNF-aR2, interleukin (IL)-2, IL-2R, IL-4, IL-6, IL-8, IL-10, and interferon-α, have been described in patients with ACLF.

Hyperbilirubinemia is almost invariably present and jaundice is considered an essential criterion of AD and ACLF. Various authors have used different cutoff levels of jaundice, varying from a serum bilirubin of 6-20 mg/dL. Besides jaundice, another hallmark of liver dysfunction is coagulopathy. Coagulation tests are usually abnormal in cirrhotic patients due to impaired synthesis and increased consumption of coagulation factors. Ongoing liver injury culminates in an inexorable downward spiral and death.

The most common organ to fail besides liver is the kidney. Renal failure may be categorized into four types: hepatorenal syndrome, parenchymal disease, hypovolemia-induced and drug-induced renal failure. Bacterial infection (such as spontaneous bacterial peritonitis) is the most common precipitating cause of renal failure in cirrhosis, followed by hypovolemia (secondary to gastrointestinal bleeding, excessive diuretic treatment).

HE is one of the common manifestations of AD and ACLF. HE may be a precipitating factor or a consequence of AD and ACLF. Ammonia is central to the pathogenesis of HE. Indeed, multiple studies have highlighted that hyperammonemia plays a critical role in the development of HE in patients with liver cirrhosis and other liver diseases. Due to liver failure, a large amount of serum ammonia escapes liver metabolism and can reach brain where such high ammonia concentrations are closely related to a high incidence of cerebral edema and herniation. In addition, brain swelling is an important feature of AD and ACLF, similar to the situation in ALF.

One of the hallmark of AD and ACLF is cardiovascular collapse akin to that in patients with ALF. This cardiovascular abnormality is associated with an increased risk of death, particularly in those patients who present renal dysfunction.

Respiratory complications in AD and ACLF can be categorized as acute respiratory failure (e.g., pneumonia) and those that arise as a consequence of cirrhosis (e.g., portopulmonary hypertension and hepatopulmonary syndrome). Patients with cirrhosis are at increased risk of pneumonia.

Patients with AD and ACLF have a statistically higher mortality rate at the same MELD score than patients without ACLF. Regardless of the precipitating event, the final common pathway leading to acute deterioration of liver function and multiorgan failure appears to be an exaggerated activation of systemic inflammation, which is then followed by a period of immune system paralysis. The initial cytokine storm is responsible for profound alterations in macrocirculation, microcirculation, and disruption of normal organ function, resulting in multiorgan failure.

Early interventions to reduce or correct injury are crucial. For patients with more than 3 organ failures, management of ACLF is currently based on the supportive treatment of organ failures, mainly in an intensive care setting. However, the proportion of cases with previous episodes of acute decompensation (development of ascites, encephalopathy, gastrointestinal hemorrhage, bacterial infection) is very frequent in patients with ACLF. Indeed, the appearance of liver failure in a patient with cirrhosis represents a decisive time point in terms of medical management since this condition is frequently associated with rapidly evolving multi-organ dysfunction. The lack of liver detoxification, metabolic and regulatory functions and altered immune response lead to life-threatening complications, such as renal failure, increased susceptibility to infection, hepatic coma and systemic hemodynamic dysfunction. Moreover, only 20% of patients with advanced cirrhosis can be treated with liver transplantation. There is a need for an adequate treatment of liver failure, in particular of AD, ACLF, ALF and decompensated cirrhosis.

### SUMMARY OF THE INVENTION

The present invention relates to a PPAR agonist selected from lanifibranor, bezafibrate, fenofibrate, pemafibrate, seladelpar, saroglitazar, pioglitazone, rosiglitazone and a compound of formula (I) as defined below, or a pharmaceutically acceptable salt of a compound of formula (I), for use in a method for the treatment of liver failure in a subject in need thereof. In a particular embodiment, the invention relates to a PPAR agonist selected from lanifibranor, bezafibrate, pemafibrate, seladelpar, saroglitazar, pioglitazone, rosiglitazone and a compound of formula (I) as defined below, or a pharmaceutically acceptable salt of a compound of formula (I), for use in a method for the treatment of liver failure in a subject in need thereof.

In a particular embodiment, the PPAR agonist is selected from the following compounds, or pharmaceutically acceptable salt thereof:
2-[4-(3-methoxy-3-(4-(trifluoromethoxy)phenyl)propyl)-2,6-dimethylphenoxy]-2-methylpropanoic acid (cpd. 1);
2-[2,6-dimethyl-4-[3-[4-(trifluoromethyloxy)phenyl]-3-oxo-propyl]phenoxy]-2-methylpropanoic acid (cpd. 2);
2-[2,6-dimethyl-4-[3-[4-(trifluoromethyl)phenyl]-3-oxo-propyl]phenoxy]-2-methylpropanoic acid (cpd. 3);
2-[2,6-dimethyl-4-[3-[4-(methylthio)phenyl]-3-methoxypropyl]phenoxy]-2-methylpropanoic acid (cpd. 4);
2-[2,6-dimethyl-4-[3-[4-(methylthio)phenyl]-3-isopropyloxypropyl]phenoxy]-2-methylpropanoic acid (cpd. 5);
2-(4-(3-hydroxy-3-(4-(trifluoromethoxy)phenyl)propyl)-2,6-dimethylphenoxy)-2-methylpropanoic acid (cpd. 6);
2-(4-(3-(methoxyimino)-3-(4-(trifluoromethoxy)phenyl)propyl)-2,6-dimethylphenoxy)-2-methylpropanoic acid (cpd. 7);
2-(2-chloro-4-(3-(4-methyl-2-(4-(trifluoromethyl)phenyl)-thiazol-5-yl)-3-oxopropyl)phenoxy)-2-methylpropanoic acid (cpd. 8);
2-(2,3-dichloro-4-(3-ethoxy-3-(4-methyl-2-(4-(trifluoromethyl)-phenyl)thiazol-5-yl)propyl)phenoxy)-2-methylpropanoic acid (cpd. 9);
2-(4-(3-(benzyloxy)-3-(5-(4-(trifluoromethyl)phenyl)thien-2-yl)propyl)-2,3-dichlorophenoxy)-2-methylpropanoic acid (cpd. 10);
2-(2,3-dichloro-4-(3-methoxy-3-(5-(4-(trifluoromethyl)phenyl)-thien-2-yl)propyl)phenoxy)-2-methylpropanoic acid (cpd. 11);
2-(4-(3-methoxy-3-(4-(trifluoromethyl)phenyl)propyl)-2,6-dimethylphenoxy)-2-methylpropanoic acid (cpd. 13); and
2-[2,6-dimethyl-4-[3-[4-bromophenyl]-3-methoxypropyl]phenoxy]-2-methylpropanoic acid; or a pharmaceutically acceptable salt thereof (cpd. 14).

In a further particular embodiment, the compound is cpd. 1 or a pharmaceutically acceptable salt thereof.

In a particular embodiment, the PPAR agonist is for use in the treatment of a liver failure selected from acute decompensation (AD), on chronic liver failure (ACLF), acute liver failure (ALF) and decompensated cirrhosis.

In a particular embodiment, the PPAR agonist is for use in the treatment of AD.

In another particular embodiment, the PPAR agonist is for use in the treatment of decompensated cirrhosis.

More particularly, the PPAR agonist is for use in the treatment of ACLF.

In another embodiment, the PPAR agonist is administered to a subject having AD, decompensated cirrhosis with or without ACLF, or is at risk of AD and ACLF.

In another embodiment, the PPAR agonist is administered to a subject having decompensated cirrhosis or who is at risk of decompensated cirrhosis or acute decompensation.

In a particular embodiment, the PPAR agonist is for use in the prevention of decompensated cirrhosis.

In yet another embodiment, the PPAR agonist is for use in a method for the reversion of decompensated cirrhosis to compensated cirrhosis.

According to another embodiment, the PPAR agonist is for use in a method for the prevention of liver decompensation in a subject having ACLF.

In another embodiment, the PPAR agonist is for use in the treatment of ALF.

In another embodiment, the PPAR agonist is for use in the prevention of kidney failure or in the prevention of hepatic encephalopathy.

According to a particular embodiment, the PPAR agonist is administered to a subject having ACLF without kidney failure, or to a subject having ACLF with a non-kidney organ failure with kidney dysfunction.

According to another embodiment, the PPAR agonist is for use in the treatment of sepsis-associated ACLF.

### DESCRIPTION OF THE FIGURES

FIG. 1: Compounds according to the invention reduce TNFα and MCP1 secretion in PMA-stimulated THP1 monocytes.

FIGS. 1A and 1B show the effect of Cpd.1 on the reduction of TNFα and MCP1 secretion respectively in PMA-stimulated THP1.

For FIGS. 1A and 1B: #, ##, ### for p<0.05, p<0.01, p<0.001 compared to the vehicle (Veh) using ANOVA and Fisher's LSD test for multiple comparisons; *, **, *** for p<0.05, p<0.01, p<0.001 compared to the vehicle (Veh) using non-parametric Kruskall Wallis test with uncorrected Dunn's test fir multiple comparisons. PMA dose: 100 ng/mL.

FIG. 2: Compounds according to the invention reduce cytokine production by THP1 differentiated macrophages.

FIG. 2A shows the effect of Cpd.1 on the reduction of TNFα production by THP1 differentiated macrophages. # for p<0.05 using non-parametric Dunn's test for multiple comparison between Cpd.3 and the vehicle (Veh).

FIG. 2B shows the effect of Cpd.1 on the reduction of MCP1 production by THP1 differentiated macrophages. ### for p<0.001 using non-parametric Dunn's test for multiple comparison between Cpd.3 and the vehicle (Veh).

FIG. 3: Reduction of serum cytokine concentration in response to LPS in rats.

FIGS. 3A and 3B shows the effect of Cpd.1 on the reduction of serum IL6 and IL1β concentration respectively in response to LPS in rats. #, ##, ### for p<0.05, p<0.01, p<0.001 using Student T test. $ for p<0.05 using non-parametric Mann-Whitney test.

FIG. 4: Effect of Cpd.1 and Cpd.19 on hepatic function and cytokine level in a model of endotoxemia.

Rats were treated with Cpd.1 (3 mg/kg), Cpd.19 (100 mg/kg) or a vehicle (Veh.) every day for 3 days before LPS injection. Blood was collected 3 h after LPS injection for the measurement of total bilirubin (A), serum albumin (B) and TNFα (C) in the serum. For A-B, One-way Anova with Dunnett test for multiple testing was used to assess statistical significance. For C, One-way Anova was used to assess statistical significance. *** p<0.001, *p<0.05.

FIG. 5: Effect of Cpd.1 on hepatic expression of inflammatory genes in a model of acute liver failure.

Mice were treated with 3 mg/kg Cpd.1 or vehicle (Veh.) every day for 3 days before LPS/GaIN injection. Liver tissues were collected 4 h after LPS/GaIN injection. RT-qPCR data show the changes in the expression of genes encoding cytokines (FIGS. 5A and 5B) or Cd68 immune cell markers (FIG. 5C). mRNA levels were normalized to the expression of Rplp0 and referred to the expression measured in the untreated condition. One-way Anova with Dunnett test for multiple testing was used to assess statistical significance. *** p<0.001, ** p<0.01.

FIG. 6: Effect of Cpd.1 and Cpd.18 on circulating proinflammatory cytokines in a model of acute liver failure.

Mice were treated with 3 mg/kg Cpd.1, 1 mg/kg Cpd.18 or vehicle (Veh.) every day for 3 days before LPS/GaIN injection. Blood samples were collected 4 h after LPS/GaIN injection for the measurement of serum cytokines level. One-way Anova with Dunnett test for multiple testing was used to assess statistical significance. ** p<0.01, ***p<0.001;

FIG. 7: Effect of Cpd.18 on serum albumin level in a model of acute liver failure.

Mice were treated with 1 mg/kg Cpd.18 or vehicle (Veh.) every day for 3 days before LPS/GaIN injection. Blood samples were collected 4 h after LPS/GaIN injection for the measurement of serum albumin level. One-way Anova was used to assess statistical significance. *p<0.05.

FIG. 8: Effect of Cpd.1 on survival rate in a model of sepsis.

Cecal ligation and puncture surgery (CLP) was performed in mice at 0 h. Cpd.1 or vehicle was administrated at 0.3 mg/kg, p.o. for three days before CLP surgery and the mice were monitored for survival during 7 days (168 days). Mice found dead in the morning are counted with those from the afternoon of the day before. Statistical difference between the experimental groups was determined by using Gehan-Breslow-Wilcoxon test. *p<0.0332.

FIG. 9: Effect of Cpd.14 on MCP1 secretion induced by LPS in THP1 macrophages.

After differentiation into macrophages, THP1 cells were treated for 24 h with 1 or 10 μM of indicated Cpd.14 before stimulation for 6 h with LPS from Klebsiella. The % inhibition of MCP1 secretion was calculated over the mean LPS-vehicle condition (Veh.). Student t-test was used to assess statistical significance. Grey boxes depict significant values (p<0.05).

FIG. 10: Effect of Cpd.14 on TNFα secretion induced by LPS in THP1 macrophages.

After differentiation into macrophages, THP1 cells were treated for 24 h with 1 or 10 μM of the indicated Cpd. before stimulation for 6 h with LPS from Klebsiella. The % inhibition of TNFα secretion was calculated over the mean LPS-vehicle condition (Veh.). Student t-test was used to assess statistical significance. Grey boxes depict significant values (p<0.05).

FIG. 11: Effect of Cpd.1 on staurosporin-induced apoptosis in HepG2 cells.

HepG2 cells were pre-treated with the indicated Cpd.1 at 0.3 μM to 10 μM for 16 h before incubation of 10 μM staurosporin for additional 4 hours. Apoptosis was assessed through caspase 3/7 activity measurement. The % inhibition of caspase 3/7 activity was calculated over the mean staurosporin-vehicle condition (Veh.). Student t-test was used to assess statistical significance. Grey boxes depict significant values (p<0.05).

### DETAILED DESCRIPTION OF THE INVENTION

The present invention relates to a PPAR agonist selected from lanifibranor, bezafibrate, fenofibrate, pemafibrate, seladelpar, saroglitazar, pioglitazone, rosiglitazone and a compound of formula (I) as defined below, or a pharmaceutically acceptable salt of a compound of formula (I), for use in a method for the treatment of a liver failure.

### Definitions

In the context of the present invention, the terms below have the following meanings.

The terms mentioned herein with prefixes such as for example C1-C6, can also be used with lower numbers of carbon atoms such as C1-C2. If, for example, the term C1-C6 is used, it means that the corresponding hydrocarbon chain may comprise from 1 to 6 carbon atoms, especially 1, 2, 3, 4, 5, or 6 carbon atoms. If, for example, the term C1-C3 is used, it means that the corresponding hydrocarbon chain may comprise from 1 to 3 carbon atoms, especially 1, 2, or 3 carbon atoms.

The term “alkyl” refers to a saturated, linear or branched aliphatic group. The term “(C1-C6)alkyl” more specifically means methyl, ethyl, propyl, isopropyl, butyl, pentyl, or hexyl. In a preferred embodiment, the “alkyl” is a methyl.

The term “alkoxy” or “alkyloxy” corresponds to the alkyl group as above defined bonded to the molecule by an —O— (ether) bond. (C1-C6)alkoxy includes methoxy, ethoxy, propyloxy, isopropyloxy, butyloxy, pentyloxy, or hexyloxy. In a preferred embodiment, the “alkoxy” or “alkyloxy” is a methoxy, an ethoxy, a propoxy, an isopropyloxy, more preferably a methoxy.

The term “alkylthio” corresponds to the alkyl group as above defined bonded to the molecule by an —S— (thioether) bond. (C1-C6)alkylthio includes thiomethyl, thioethyl, thiopropyl, thioisopropyl, thiobutyl, thiopentyl, or thiohexyl. In a preferred embodiment, the “alkylthio” is a thiomethyl, a thioethyl, a thiopropyl, a thioisopropyl, more preferably a thiomethyl.

A “cyclic” group corresponds to an aryl group, a cycloalkyl group or a heterocyclic group.

The term “aryl” corresponds to a mono- or bi-cyclic aromatic hydrocarbons having from 6 to 12 carbon atoms. For instance, the term “aryl” includes phenyl, naphthyl, or anthracenyl. In a preferred embodiment, the aryl is a phenyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, preferably cyclopropyl. The term “spirocycloalkyl” includes for instance a spirocyclopropyl.

The term “cycloalkoxy” corresponds to the cycloalkyl group as above defined bonded to the molecule by an —O— (ether) bond.

The term “cycloalkylthio” corresponds to the cycloalkyl group as above defined bonded to the molecule by an —S— (thioether) bond.

The term “heterocycloalkyl” corresponds to a saturated or unsaturated cycloalkyl group as above defined further comprising at least one heteroatom such as nitrogen, oxygen, or sulphur atom, preferably at least one nitrogen atom. It also includes fused, bridged, or spiro-connected heterocycloalkyl groups. Representative heterocycloalkyl groups include, but are not limited to dioxolanyl, benzo[1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, dithiolanyl, azepanyl, thiazolinyl, thiazolidinyl, isothiazolinyl, isothiazolidinyl, dihydropyranyl, tetrahydropyranyl, tetrahydrofuranyl, and tetrahydrothiophenyl. In a preferred embodiment, the heterocycloalkyl group is morpholinyl, piperazinyl, piperidinyl, pyrrolidinyl, tetrahydropyranyl, dithiolanyl and azepanyl groups, more preferably piperidinyl.

The term “heteroaryl” as used herein corresponds to an aromatic, mono- or poly-cyclic group comprising between 5 and 14 atoms and comprising at least one heteroatom such as nitrogen, oxygen or sulphur atom. As used herein, the term “heteroaryl” further includes the “fused arylheterocycloalkyl” and “fused heteroarylcycloalkyl”. The terms “fused arylheterocycloalkyl” and “fused heteroarylcycloalkyl” correspond to a bicyclic group in which an aryl as above defined or a heteroaryl is respectively bounded to the heterocycloalkyl or the cycloalkyl as above defined by at least two carbons. In other terms, the aryl or the heteroaryl respectively shares a carbon bond with the heterocycloalkyl or the cycloalkyl. Examples of such mono- and poly-cyclic heteroaryl groups may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolyl, benzoxazolinyl, benzoxazinyl, benzothienyl, benzothiazolyl, benzodiazepinyl, benzazepinyl, benzoxazepinyl, isatinyl, dihydropyridyl, pyrimidinyl, s-triazinyl, oxazolyl, or thiofuranyl. In a preferred embodiment, a heteroaryl is a thiazolyl, pyridinyl, pyrimidinyl, furanyl, thiophenyl, quinolinyl, and isoquinolinyl, more preferably a thiazolyl and thiophenyl.

The term “heterocyclic” refers to a heterocycloalkyl group or a heteroaryl group as above defined.

The term “halogen” corresponds to a fluorine, chlorine, bromine, or iodine atom, preferably a fluorine atom, a chlorine atom or a bromine atom.

The term “pharmaceutically acceptable salts” includes inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. The “pharmaceutically acceptable salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheeps and non-human primates, among others.

The expression “substituted by at least” means that the radical is substituted by one or several groups of the list.

In the context of the present invention, the term “about” applied to a numerical value means the value+/−10%. For the sake of clarity, this means that “about 100” refers to values comprised in the 90-110 range. In addition, in the context of the present invention, the term “about X”, wherein X is a numerical value, also discloses specifically the X value, but also the lower and higher value of the range defined as such, more specifically the X value.

### Compounds for Use in the Present Invention

The present invention provides a PPAR agonist selected from lanifibranor, bezafibrate, fenofibrate, pemafibrate, seladelpar, saroglitazar, pioglitazone, rosiglitazone and a compound of formula (I) as defined below, or a pharmaceutically acceptable salt of a compound of formula (I), for use in a method for the treatment of liver failure. In yet another particular embodiment, the invention provides a PPAR agonist selected from lanifibranor, bezafibrate, pemafibrate, seladelpar, saroglitazar, pioglitazone, rosiglitazone and a compound of formula (I) as defined below, or a pharmaceutically acceptable salt of a compound of formula (I), for use in a method for the treatment of liver failure.

In a particular embodiment, the PPAR agonist is selected from lanifibranor, bezafibrate, fenofibrate, pemafibrate, seladelpar, saroglitazar, pioglitazone and rosiglitazone. In a further particular embodiment, the PPAR agonist is selected from lanifibranor, bezafibrate, pemafibrate, seladelpar, saroglitazar, pioglitazone and rosiglitazone.

In another particular embodiment, the PPAR agonist for use according to the invention is a compound of formula (I), or a pharmaceutically acceptable salt thereof,

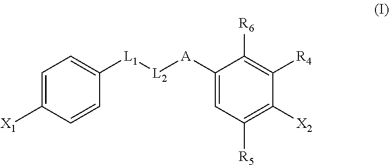

wherein:
X1 represents a halogen atom, a R1 group or a G1-R1 group;
L1 represents a bond, a thiophenyl group or a thiazole group substituted or not by a (C1-C3)alkyl group;
L2 represents:
(i) a —CH—OR7 group, in which R7 represents a hydrogen atom, an unsubstituted (C1-C6)alkyl group or a (C1-C6)alkyl group substituted by a (C6-C14)aryl group, in particular in which R7 represents an unsubstituted (C1-C6)alkyl group or a (C1-C6)alkyl group substituted by a (C6-C14)aryl group;
(ii) a carbonyl group (CO); or
(iii) a C═N—OR8, in which R8 represents an unsubstituted (C1-C6)alkyl group;
A represents a CH═CH or a CH₂—CH₂ group;
X2 represents a G2-R2 group;
G1 and G2, identical or different, represent an atom of oxygen or sulfur;
R1 represents a hydrogen atom, an unsubstituted (C1-C6)alkyl group, a (C6-C14)aryl group or an alkyl group that is substituted by at least one substituent selected from halogen atoms, (C1-C6)alkoxy groups, (C1-C6)alkylthio groups, (C5-C10)cycloalkyl groups, (C5-C10)cycloalkylthio groups and 5- to 14-membered heterocyclic groups;
R2 represents a (C1-C6)alkyl group substituted by a —COOR3 group;
R3 represents a hydrogen atom or a (C1-C6)alkyl group that is substituted or not by at least one substituent selected from halogen atoms, (C5-C10)cycloalkyl groups and 5- to 14-membered heterocyclic groups;
R4 represents a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups;
R5 represents a hydrogen atom, a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups; and R6 represents a hydrogen atom or a halogen atom;
with the proviso that the compound of formula (I) is not:
   elafibranor or a pharmaceutically acceptable salt thereof; or
   2-[2,6-dimethyl-4-[3-[4-(methylthio)phenyl]-3-oxo-propyl]phenoxy]-2-methylpropanoic acid or a pharmaceutically acceptable salt thereof.

In a particular embodiment, L1 represents a bond, and R6 is a hydrogen atom, i.e. the compound of formula (I) is a compound of formula (Ia) as represented below:

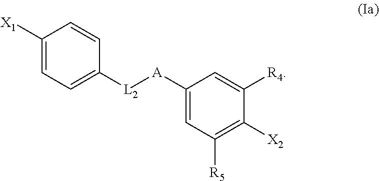

In another embodiment, L1 represents a thiazol group that is substituted or not by a (C1-C3)alkyl group, in particular by a methyl group. In a particular embodiment, L1 represents a 2-methyl-thiazolyl group. In a further particular embodiment, L1 is a 2-methyl-thiazolyl group and the compound of formula (I) is a compound of formula (Ib) as represent below:

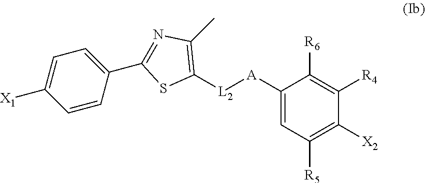

In another particular embodiment, L1 represents a thiophenyl group. In yet another embodiment, L1 represents a thiophenyl group and the compound of formula (I) is a compound of formula (Ic) as represented below:

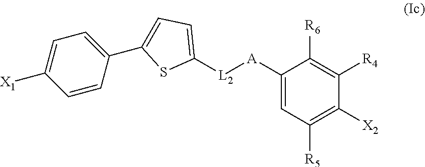

In a particular embodiment, X1 is a R1 group wherein R1 is an unsubstituted (C1-C6)alkyl group or a (C1-C6)alkyl group that is substituted by one or more halogen atoms.

In another particular embodiment, X1 is a R1 group wherein R1 is an unsubstituted (C1-C6)alkyl group. In another particular embodiment, X1 is a R1 group wherein R1 is an unsubstituted (C1-C4)alkyl group. In another particular embodiment, X1 is a R1 group wherein R1 is an unsubstituted (C1-C3)alkyl group. In another particular embodiment, X1 is a R1 group wherein R1 is a methyl or ethyl group. In another particular embodiment, X1 is a R1 group wherein R1 is a methyl group.

In another particular embodiment, X1 is a R1 group wherein R1 is a (C1-C6)alkyl group that is substituted by one or more halogen atoms. In another particular embodiment, X1 is a R1 group wherein R1 is a (C1-C4)alkyl group that is substituted by one or more halogen atoms. In another particular embodiment, X1 is a R1 group wherein R1 is a (C1-C3)alkyl group that is substituted by one or more halogen atoms. In another particular embodiment, X1 is a R1 group wherein R1 is a methyl or ethyl group substituted by one or more halogen atoms. In another particular embodiment, X1 is a R1 group wherein R1 is a methyl group substituted by one or more halogen atoms. In another particular embodiment, X1 is a R1 group wherein R1 is a trifluoromethyl group.

In a particular embodiment, G1 is a sulfur atom.

In another particular embodiment, G1 is a sulfur atom and R1 is a (C1-C6)alkyl group that is substituted by one or more halogen atoms. In another particular embodiment, G1 is a sulfur atom and R1 is a (C1-C4)alkyl group that is substituted by one or more halogen atoms. In another particular embodiment, G1 is a sulfur atom and R1 is a (C1-C3)alkyl group that is substituted by one or more halogen atoms. In another particular embodiment, G1 is a sulfur atom and R1 is a methyl or ethyl group substituted by one or more halogen atoms. In another particular embodiment, G1 is a sulfur atom and R1 is a methyl group substituted by one or more halogen atoms. In another particular embodiment, G1 is a sulfur atom and R1 is a trifluoromethyl group.

In a further particular embodiment, G1 is a sulfur atom and R1 is an unsubstituted (C1-C6)alkyl group or a (C1-C6)alkyl group that is substituted by one or more halogen atoms. In another particular embodiment, G1 is a sulfur atom and R1 is an unsubstituted (C1-C6)alkyl group. In another particular embodiment, G1 is a sulfur atom and R1 is an unsubstituted (C1-C4)alkyl group. In another particular embodiment, G1 is a sulfur atom and R1 is an unsubstituted (C1-C3)alkyl group. In another particular embodiment, G1 is a sulfur atom and R1 is a methyl or ethyl group. In another particular embodiment, G1 is a sulfur atom and R1 is a methyl group.

In yet another particular embodiment, G1 is an oxygen atom.

In a further particular embodiment, G1 is an oxygen atom and R1 is an unsubstituted (C1-C6)alkyl group or a (C1-C6)alkyl group that is substituted by one or more halogen atoms. In another particular embodiment, G1 is an oxygen atom and R1 is an unsubstituted (C1-C6)alkyl group. In another particular embodiment, G1 is an oxygen atom and R1 is an unsubstituted (C1-C4)alkyl group. In another particular embodiment, G1 is an oxygen atom and R1 is an unsubstituted (C1-C3)alkyl group. In another particular embodiment, G1 is an oxygen atom and R1 is a methyl or ethyl group. In another particular embodiment, G1 is an oxygen atom and R1 is a methyl group.

In another particular embodiment, G1 is an oxygen atom and R1 is a (C1-C6)alkyl group that is substituted by one or more halogen atoms. In another particular embodiment, G1 is an oxygen atom and R1 is a (C1-C4)alkyl group that is substituted by one or more halogen atoms. In another particular embodiment, G1 is an oxygen atom and R1 is a (C1-C3)alkyl group that is substituted by one or more halogen atoms. In another particular embodiment, G1 is an oxygen atom and R1 is a methyl or ethyl group substituted by one or more halogen atoms. In another particular embodiment, G1 is an oxygen atom and R1 is a methyl group substituted by one or more halogen atoms. In another particular embodiment, G1 is an oxygen atom and R1 is a trifluoromethyl group.

In another particular embodiment, G2 is an oxygen atom.

In a further particular embodiment, R2 represents a (C1-C4)alkyl group that is substituted by a —COOR3 group. In another embodiment, R2 represents a (C1-C3)alkyl group that is substituted by a COOR3 group. In another embodiment, R2 represents a C(CH₃)₂ group substituted by a —COOR3 group.

In another particular embodiment, R3 is a hydrogen atom or an unsubstituted (C1-C6)alkyl group. In another embodiment, R3 is a hydrogen atom or an unsubstituted (C1-C4)alkyl group.

In another embodiment, R3 is a hydrogen atom or methyl, ethyl, propyl, isopropyl, butyl, n-butyl, isobutyl or tertbutyl group. In another particular embodiment, R3 is a hydrogen atom.

In another particular embodiment, R4 is a halogen atom or an unsubstituted (C1-C6)alkyl group. In another embodiment, R4 is a chlorine atom. In another embodiment, R4 is an unsubstituted (C1-C6)alkyl group. In another embodiment, R4 is an unsubstituted (C1-C4)alkyl group. In another embodiment, R4 is an unsubstituted (C1-C3)alkyl group. In another embodiment, R4 is a methyl or ethyl group. In another embodiment, R4 is a methyl group.

In another particular embodiment, R5 is hydrogen atom or an unsubstituted (C1-C6)alkyl group. In a particular embodiment, R5 is a hydrogen atom. In another embodiment, R5 is an unsubstituted (C1-C6)alkyl group. In another embodiment, R5 is an unsubstituted (C1-C4)alkyl group. In another embodiment, R5 is an unsubstituted (C1-C3)alkyl group. In another embodiment, R5 is a methyl or ethyl group. In another embodiment, R5 is a methyl group.

In another particular embodiment, R4 and R5 are identical. In another embodiment, R4 and R5 are an unsubstituted (C1-C6)alkyl group. In another embodiment, R4 and R5 are an unsubstituted (C1-C6)alkyl group. In another embodiment, R4 and R5 are an unsubstituted (C1-C4)alkyl group. In another embodiment, R4 and R5 are an unsubstituted (C1-C3)alkyl group. In another embodiment, R4 and R5 are a methyl or ethyl group. In another embodiment, R4 and R5 are a methyl group.

In a particular embodiment, R6 is a halogen atom. In another embodiment, R6 is a chlorine atom.

In another particular embodiment, R4 and R6 are identical. In another embodiment, R4 and R6 are a halogen atom. In another embodiment, R4 and R6 are chlorine atom.

In another particular embodiment, L2 is a —CH—OR7 group. In another embodiment, R7 is an unsubstituted (C1-C4)alkyl group. In another embodiment, R7 is an unsubstituted (C1-C3)alkyl group. In another embodiment, R7 is a methyl or ethyl group. In another embodiment, R7 is a methyl group.

In a particular embodiment, R7 is a (C1-C6)alkyl substituted by a phenyl group. In another particular embodiment, R7 is a methyl or ethyl group substituted by a phenyl group. In yet another embodiment, R7 is a benzyl group.

In another particular embodiment, L2 is a carbonyl group.

In another particular embodiment, L2 is a C═N—OR8. In another embodiment, R8 is an unsubstituted (C1-C4)alkyl group. In another embodiment, R8 is an unsubstituted (C1-C3)alkyl group. In another embodiment, R8 is a methyl or ethyl group. In another embodiment, R8 is a methyl group.

In another particular embodiment, the PPAR agonist is a compound of formula (Ia) wherein:
G1 is a sulfur atom;
R1 is an unsubstituted (C1-C4)alkyl group;
R2 is a (C1-C3)alkyl group substituted by a —COOR3 group;
R3 is a hydrogen atom or an unsubstituted (C1-C4)alkyl group;
R4 and R5 represent a (C1-C4)alkyl group; and
L2 is a carbonyl group.

In another particular embodiment, the PPAR agonist is a compound of formula (Ia) wherein:
G1 is a sulfur atom;
R1 is an unsubstituted (C1-C4)alkyl group;
R2 is a C(CH₃)₂ group substituted by a —COOR3 group;
R3 is a hydrogen atom or an unsubstituted (C1-C4)alkyl group;
R4 and R5 represent a (C1-C4)alkyl group; and
L2 is a carbonyl group.

In another particular embodiment, the PPAR agonist is a compound of formula (Ia) wherein:
G1 is a sulfur atom;
R1 is an unsubstituted (C1-C4)alkyl group;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a carbonyl group; and
A is a CH═CH group.

In another particular embodiment, the PPAR agonist is a compound of formula (Ia) wherein:
G1 is a sulfur atom;
R1 is an unsubstituted (C1-C4)alkyl group;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a carbonyl group; and
A is a CH₂—CH₂ group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group; and
L2 is a —CH—OR7 group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group;
L2 is a —CH—OR7 group; and
R7 is an unsubstituted (C1-C4)alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group; and
R7 is an unsubstituted (C1-C4)alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group;
R7 is an unsubstituted (C1-C4)alkyl group; and
A is a CH═CH group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group;
R7 is an unsubstituted (C1-C4)alkyl group; and
A is a CH₂—CH₂ group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group;
R7 is a (C1-C6)alkyl group substituted by a (C6-C14)aryl group; and
A is a CH═CH group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group;
L2 is a C═N—OR8 group; and
R8 represents an unsubstituted (C1-C6) alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a C═N—OR8 group; and
R8 is an unsubstituted (C1-C4)alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a C═N—OR8 group;
R8 is an unsubstituted (C1-C4)alkyl group; and
A is a CH═CH group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a C═N—OR8 group;
R8 is an unsubstituted (C1-C4)alkyl group; and
A is a CH₂—CH₂ group.

In another particular embodiment, the PPAR agonist is a compound of formula (Ib) wherein X1 is a R1 group. In another particular embodiment, the PPAR agonist is a compound of formula (Ib) wherein X1 is a R1 group wherein R1 is a (C1-C6)alkyl group substituted by at least one halogen atoms. In another particular embodiment, the PPAR agonist is a compound of formula (Ib) wherein X1 is a R1 group wherein R1 is a CF3 group.

In another particular embodiment, the PPAR agonist is a compound of formula (Ib) wherein L2 represents a —CH—OR7 group or a carbonyl group. In yet another embodiment, L2 represents a carbonyl group in formula (Ib).

In a further particular embodiment, the PPAR agonist is a compound of formula (Ib) wherein R4 is a halogen atom. In yet another embodiment, R4 is a chlorine atom in formula (Ib).

In a further particular embodiment, the PPAR agonist is a compound of formula (Ib) wherein R5 is a hydrogen atom.

In another embodiment, the PPAR agonist is a compound of formula (Ib) wherein R6 is a hydrogen atom.

In another embodiment, the PPAR agonist is a compound of formula (Ib) wherein R6 is a halogen atom.

In another embodiment, the PPAR agonist is a compound of formula (Ib) wherein R4 and R6 are halogen atoms. In another embodiment, the PPAR agonist is a compound of formula (Ib) wherein R4 and R6 are the halogen atom. In another embodiment, the PPAR agonist is a compound of formula (Ic) wherein R4 and R6 are a chlorine atom

In another embodiment, the PPAR agonist is a compound of formula (Ib) wherein R2 is a (C1-C3)alkyl group substituted by a —COOR3 group. In another embodiment, the PPAR agonist is a compound of formula (Ib) wherein R2 is a C(CH₃)₂ group substituted by a —COOR3 group.

In another embodiment, the PPAR agonist is a compound of formula (Ib) wherein R3 is a hydrogen atom or a (C1-C4)alkyl group. In yet another embodiment, R3 is a hydrogen atom.

In another embodiment, the PPAR agonist is a compound of formula (Ib) wherein A is a CH₂—CH₂ group.

In another particular embodiment, the PPAR agonist is a compound of formula (Ib) wherein:
X1 is a R1 group;
R1 is a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 represents a halogen atom;
R5 represents a hydrogen atom;
R6 represents a hydrogen atom;
L2 is a carbonyl group; and
A is a CH₂—CH₂ group.

In another particular embodiment, the PPAR agonist is a compound of formula (Ib) wherein:
X1 is a R1 group;
R1 is a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 represents a halogen atom;
R5 represents a hydrogen atom;
R6 represents a halogen atom;
L2 is a carbonyl group; and
A is a CH₂—CH₂ group.

In another particular embodiment, the PPAR agonist is a compound of formula (Ic) wherein X1 is a R1 group. In another particular embodiment, the PPAR agonist is a compound of formula (Ic) wherein X1 is a R1 group wherein R1 is a (C1-C6)alkyl group substituted by at least one halogen atoms. In another particular embodiment, the PPAR agonist is a compound of formula (Ic) wherein X1 is a R1 group wherein R1 is a CF3 group.

In another particular embodiment, the PPAR agonist is a compound of formula (Ic) wherein L2 represents a —CH—OR7 group.

In another embodiment, the PPAR agonist is a compound of formula (Ic) wherein R7 is an unsubstituted (C1-C4)alkyl group or a (C1-C4)alkyl group substituted by a (C6-C14)aryl group.

In another embodiment, the PPAR agonist is a compound of formula (Ic) wherein R7 is a methyl group or a benzyl group.

In a further particular embodiment, the PPAR agonist is a compound of formula (Ic) wherein R4 is a halogen atom. In yet another embodiment, R4 is a chlorine atom in formula (Ic).

In a further particular embodiment, the PPAR agonist is a compound of formula (Ic) wherein R5 is a hydrogen atom.

In another embodiment, the PPAR agonist is a compound of formula (Ic) wherein R6 is a halogen atom.

In another embodiment, the PPAR agonist is a compound of formula (Ic) wherein R4 and R6 are halogen atoms. In another embodiment, the PPAR agonist is a compound of formula (Ic) wherein R4 and R6 are the halogen atom. In another embodiment, the PPAR agonist is a compound of formula (Ic) wherein R4 and R6 are a chlorine atom

In another embodiment, the PPAR agonist is a compound of formula (Ic) wherein R2 is a (C1-C3)alkyl group substituted by a —COOR3 group. In another embodiment, the PPAR agonist is a compound of formula (Ic) wherein R2 is a C(CH₃)₂ group substituted by a —COOR3 group.

In another embodiment, the PPAR agonist is a compound of formula (Ic) wherein R3 is a hydrogen atom or a (C1-C4)alkyl group. In yet another embodiment, R3 is a hydrogen atom.

In another embodiment, the PPAR agonist is a compound of formula (Ib) wherein A is a CH₂—CH₂ group.

In another particular embodiment, the PPAR agonist is a compound of formula (Ic) wherein:
X1 is a R1 group;
R1 is a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 represents a halogen atom;
R5 represents a hydrogen atom;
R6 represents a halogen atom;
L2 is a carbonyl group; and
A is a CH₂—CH₂ group.

In a particular embodiment, the compound of formula (I) is selected from:
Cpd.1: 2-[4-(3-methoxy-3-(4-(trifluoromethoxy)phenyl)propyl)-2,6-dimethylphenoxy]-2-methylpropanoic acid or a pharmaceutically acceptable salt thereof; Cpd. 2: 2-[2,6-dimethyl-4-[3-[4-(trifluoromethyloxy)phenyl]-3-oxo-propyl]phenoxy]-2-methylpropanoic acid or a pharmaceutically acceptable salt thereof;
Cpd.3: 2-[2,6-dimethyl-4-[3-[4-(trifluoromethyl)phenyl]-3-oxo-propyl]phenoxy]-2-methylpropanoic acid or a pharmaceutically acceptable salt thereof;
Cpd.4: 2-[2,6-dimethyl-4-[3-[4-(methylthio)phenyl]-3-methoxypropyl]phenoxy]-2-methylpropanoic acid or a pharmaceutically acceptable salt thereof;
Cpd.5: 2-[2,6-dimethyl-4-[3-[4-(methylthio)phenyl]-3-isopropyloxypropyl]phenoxy]-2-methylpropanoic acid or a pharmaceutically acceptable salt thereof;
Cpd.6: 2-(4-(3-hydroxy-3-(4-(trifluoromethoxy)phenyl)propyl)-2,6-dimethylphenoxy)-2-methylpropanoic acid or a pharmaceutically acceptable salt thereof;
Cpd.7: 2-(4-(3-(methoxyimino)-3-(4-(trifluoromethoxy)phenyl)propyl)-2,6-dimethylphenoxy)-2-methylpropanoic acid or a pharmaceutically acceptable salt thereof;
Cpd.8: 2-(2-chloro-4-(3-(4-methyl-2-(4-(trifluoromethyl)phenyl)-thiazol-5-yl)-3-oxopropyl)phenoxy)-2-methylpropanoic acid or a pharmaceutically acceptable salt thereof;
Cpd.9: 2-(2,3-dichloro-4-(3-ethoxy-3-(4-methyl-2-(4-(trifluoromethyl)-phenyl)thiazol-5-yl)propyl)phenoxy)-2-methylpropanoic acid or a pharmaceutically acceptable salt thereof;
Cpd.10: 2-(4-(3-(benzyloxy)-3-(5-(4-(trifluoromethyl)phenyl)thien-2-yl)propyl)-2,3-dichlorophenoxy)-2-methylpropanoic acid or a pharmaceutically acceptable salt thereof;
Cpd.11: 2-(2,3-dichloro-4-(3-methoxy-3-(5-(4-(trifluoromethyl)phenyl)-thien-2-yl)propyl)phenoxy)-2-methylpropanoic acid or a pharmaceutically acceptable salt thereof;
Cpd.13: 2-(4-(3-methoxy-3-(4-(trifluoromethyl)phenyl)propyl)-2,6-dimethylphenoxy)-2-methylpropanoic acid or a pharmaceutically acceptable salt thereof;
Cpd.14: 2-[2,6-dimethyl-4-[3-[4-bromophenyl]-3-methoxypropyl]phenoxy]-2-methylpropanoic acid or a pharmaceutically acceptable salt thereof;
Cpd.17: bezafibrate;
Cpd.18: pemafibrate;
Cpd.19: fenofibrate;
Cpd.20: seladelpar lysine;
Cpd.21: pioglitazone;
Cpd.22: rosiglitazone; and
Cpd.23: lanifibranor.

In a more particular embodiment, the compound of formula (I) is Cpd.1: 2-[4-(3-methoxy-3-(4-(trifluoromethoxy)phenyl)propyl)-2,6-dimethylphenoxy]-2-methylpropanoic acid or a pharmaceutically acceptable salt thereof.

The compound of formula (I) can be in the form of a pharmaceutically acceptable salt, particularly acid or base salts compatible with pharmaceutical use. Salts of compounds of formula (I) include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified agonist.

The present invention also relates to a pharmaceutically acceptable salt of 2-[4-(3-methoxy-3-(4-(trifluoromethoxy)phenyl)propyl)-2,6-dimethylphenoxy]-2-methylpropanoic acid. In a particular embodiment, the pharmaceutically acceptable salt of 2-[4-(3-methoxy-3-(4-(trifluoromethoxy)phenyl)propyl)-2,6-dimethylphenoxy]-2-methylpropanoic acid is the sodium, calcium, L-lysine or glycine salt thereof.

In a particular embodiment, the invention relates to the sodium salt of 2-[4-(3-methoxy-3-(4-(trifluoromethoxy)phenyl)propyl)-2,6-dimethylphenoxy]-2-methylpropanoic acid.

In a particular embodiment, the invention relates to the calcium salt of 2-[4-(3-methoxy-3-(4-(trifluoromethoxy)phenyl)propyl)-2,6-dimethylphenoxy]-2-methylpropanoic acid.

In a particular embodiment, the invention relates to the L-lysine salt of 2-[4-(3-methoxy-3-(4-(trifluoromethoxy)phenyl)propyl)-2,6-dimethylphenoxy]-2-methylpropanoic acid.

In a particular embodiment, the invention relates to the glycine salt of 2-[4-(3-methoxy-3-(4-(trifluoromethoxy)phenyl)propyl)-2,6-dimethylphenoxy]-2-methylpropanoic acid.

In another aspect, the invention relates to a PPAR agonist for use in the treatment of liver failure, wherein the PPAR agonist is selected from pharmaceutically acceptable salts of 2-[4-(3-methoxy-3-(4-(trifluoromethoxy)phenyl)propyl)-2,6-dimethylphenoxy]-2-methylpropanoic acid. In a particular embodiment, the PPAR agonist for use according to the invention is the sodium, calcium, L-lysine or glycine salt of 2-[4-(3-methoxy-3-(4-(trifluoromethoxy)phenyl)propyl)-2,6-dimethylphenoxy]-2-methylpropanoic acid.

### Liver Failure

In a particular embodiment, the subject is a patient with a liver failure selected in the group consisting of AD, ACLF, ALF and cirrhosis, such as compensated or decompensated cirrhosis.

In a particular embodiment, the subject is a patient with a liver failure selected in the group consisting of ACLF, ALF and decompensated cirrhosis.

Alternatively, the subject in need of the treatment is a subject at risk of a liver failure selected from AD, ACLF, ALF and cirrhosis. In a particular embodiment, the subject is at risk of a liver failure selected in the group consisting of AD, ACLF, ALF and decompensated cirrhosis. In particular, the subject may be a patient at risk of AD, ACLF or at risk of decompensated cirrhosis due to a chronic liver disease.

In a particular embodiment, the subject has ALF. In another embodiment, the subject has ALF caused by drug-induced liver injury, paracetamol toxicity, ischaemia, hepatitis A, B or E, autoimmunity, heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex and diffusely infiltrating malignancies. In yet another embodiment, the subject has ALF caused by drug-induced liver injury, paracetamol toxicity, ischaemia, hepatitis A, B or E, autoimmunity. In yet another embodiment, the subject has ALF caused by paracetamol toxicity.

In another particular embodiment, the subject is at risk of ALF. In another embodiment, the subject is at risk of ALF caused by drug-induced liver injury, paracetamol toxicity, ischaemia, hepatitis A, B or E, autoimmunity, heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex and diffusely infiltrating malignancies. In yet another embodiment, the subject is at risk of ALF caused by drug-induced liver injury, paracetamol toxicity, ischaemia, hepatitis A, B or E, autoimmunity. In yet another embodiment, the subject is at risk of ALF caused by paracetamol toxicity.

In a particular embodiment, the subject has compensated or decompensated cirrhosis, in particular decompensated cirrhosis. In a particular embodiment, the subject has alcoholic cirrhosis, such as alcoholic compensated cirrhosis or alcoholic decompensated cirrhosis, more particularly alcoholic decompensated cirrhosis. In another particular embodiment, the subject has compensated or decompensated cirrhosis consecutive to nonalcoholic fatty liver disease (NAFLD). In another particular embodiment, the subject has decompensated cirrhosis consecutive to nonalcoholic fatty liver disease (NAFLD). In another particular embodiment, the subject has compensated or decompensated cirrhosis consecutive to nonalcoholic steatohepatitis (NASH). In another particular embodiment, the subject has decompensated cirrhosis consecutive to nonalcoholic steatohepatitis (NASH).

In a particular embodiment, the subject is at risk of compensated or decompensated cirrhosis, in particular of decompensated cirrhosis. In a particular embodiment, the subject is at risk of alcoholic cirrhosis, such as of alcoholic compensated cirrhosis or alcoholic decompensated cirrhosis, more particularly of alcoholic decompensated cirrhosis. In another particular embodiment, the subject is at risk of compensated or decompensated cirrhosis consecutive to nonalcoholic fatty liver disease (NAFLD). In another particular embodiment, the subject is at risk of decompensated cirrhosis consecutive to nonalcoholic fatty liver disease (NAFLD). In another particular embodiment, the subject is at risk of compensated or decompensated cirrhosis consecutive to nonalcoholic steatohepatitis (NASH). In another particular embodiment, the subject is at risk of decompensated cirrhosis consecutive to nonalcoholic steatohepatitis (NASH).

In another particular embodiment, the subject has compensated or decompensated cirrhosis and is at risk of AD and ACLF. In another embodiment, the subject has decompensated cirrhosis and is at risk of AD and ACLF.

In another particular embodiment, the subject has ACLF or is at risk of ACLF.

As mentioned above, ACLF is a multiorgan syndrome that generally develops in subjects with cirrhosis, in particular in subjects with decompensated cirrhosis, with at least one organ failure and with high short-term mortality rate. ACLF can develop in patients with chronic liver disease in response to sur-imposed precipitating factors.

In a particular embodiment, the subject suffers from a chronic liver disease with cirrhosis and is at risk of developing ACLF.

The term “chronic liver disease” is used herein to refer to liver diseases associated with a chronic liver injury regardless of the underlying cause. A chronic liver disease may result, for example, from alcohol abuse (alcoholic hepatitis), from viral infectious processes (e.g. viral hepatitis A, B, C, E), autoimmune processes (autoimmune hepatitis), non-alcoholic steatohepatitis (NASH), cancer or chronic exposure to mechanical or chemical injury to the liver. Chemical injury to the liver can be caused by a variety of substances, such as toxins, alcohol, carbon tetrachloride, trichloroethylene, iron or medications.

In a particular embodiment, the subject has a chronic liver disease with cirrhosis. In a particular embodiment, the subject has cirrhosis consecutive to:
alcohol abuse,
viral hepatitis (such as a viral hepatitis resulting from hepatitis A, B, C, D, E, or G virus infection), use of medication,
metabolic disease,
a biliary disease,
primary biliary cholangitis,
primary sclerosing cholangitis, or
NASH.

The present invention is particularly suitable for the prevention of the recurrence or management of AD and ACLF.

In a particular embodiment, the subject with decompensated cirrhosis, AD or ACLF, shows a high MELD score. The term “MELD score” or “Model for End-Stage Liver Disease” as used herein refers to a scoring system for assessing the severity of liver dysfunction. MELD uses the patient's values for serum bilirubin, serum creatinine and the international ratio for prothrombin time (INR) to predict survival. It is calculated according to the following formula: MELD=3.78 [Ln serum bilirubin (mg/dL)]+11.2 [Ln INR]+9.57 [Ln serum creatinine (mg/dL)]+6.43 wherein, Ln means Napierian logarithm.

Bilirubin is the yellow breakdown product of normal heme catabolism. Bilirubin is excreted in bile and urine. Most bilirubin (70-90%) is derived from hemoglobin degradation and, to a lesser extent, from other hemoproteins. In serum, bilirubin is usually measured as both direct bilirubin and total bilirubin. Direct bilirubin correlates with conjugated bilirubin and it includes both the conjugated bilirubin and bilirubin covalently bound to albumin. Indirect bilirubin correlates to unconjugated bilirubin. The serum bilirubin level can be measured by any suitable method known in the art. Illustrative non-limitative examples of methods for determining serum bilirubin include methods using diazo reagent, methods with DPD, methods with bilirubin oxidase or by means of direct spectrophotometric determination of bilirubin. Briefly, the method for determining the levels of bilirubin in serum with diazo reagents is based on the formation of azobilirubin, which acts as indicator by means of addition of a mixture of sufanilic acid and sodium nitrite. The method based in determining serum bilirubin with DPD is based on the fact that bilirubin reacts with 2,5-dichlorobenzenediazonium salt (DPD) in 0.1 mol/HCl forming azobilirubin with maximal absorbance at 540-560 nm. The staining intensity is proportional to the concentration of bilirubin. Unconjugated bilirubin reacting in the presence of detergent (e.g. Triton TX-100) is determined as total bilirubin whereas only conjugated bilirubin reacts in the absence of detergent. The method for determining the serum level of bilirubin with bilirubin oxidase is based on the reaction catalyzed by the enzyme bilirubin oxidase which oxidizes bilirubin to biliverdin with maximal absorbance at 405-460 nm. The concentration of bilirubin is proportional to the measured absorbance. The concentration of total bilirubin is determined by the addition of sodium dodecyl sulfate (SDS) or sodium cholate which evokes the separation of unconjugated bilirubin from albumin and a reaction of precipitation. The level of serum bilirubin can also be determined by direct spectrophotometric at 454 nm and 540 nm. The measurement at these two wavelengths is used to diminish the hemoglobin interference.

The term “international ratio for prothrombin time”, or “INR” as used herein, refers to a parameter used to determine the clotting tendency of blood. The INR is the ratio of a patient's prothrombin time to a normal (control) sample, raised to the power of the ISI value for the analytical system used. Prothrombin time (PT) measures factors I (fibrinogen), II (prothrombin), V, VII and X and it is used in conjunction with the activated partial tromboplastin time. The prothrombin time is the time it takes plasma to clot after addition of tissue factor. This measures the extrinsic pathway of coagulation. The INR standardizes the results of prothrombin time and is calculated by the following formula: INR=(PTtest/PTnormal)<|S|>.

The ISI value of the formula is the International Sensitive Index for any tissue factor and it indicates how a particular batch of tissue factor compares to an international reference tissue factor. The ISI is usually between 1.0 and 2.0.

The value of MELD score correlates strongly with short-term mortality, the lower the value of MELD score the lower the mortality and the higher the value of the MELD score, the higher the mortality. Thus, a patient having low MELD score, for example a MELD lower than 9, has about 1.9% 3-month mortality whereas patients having high MELD score, for example a MELD score of 40 or more, have about 71.3% 3-month mortality.

The term “high MELD score” as used herein, refers to a patient having a MELD score higher than 9, for example, at least 10, at least 15, at least 19, at least 20, at least 25, at least 29, at least 30, at least 35, at least 39, at least 40, at least 45 or more. In a particular embodiment, the present invention is applied to a subject having a MELD score higher than 20.

In another particular embodiment, the patient to be treated shows impairment of kidney function. The term “impairment of kidney function”, also known as “impairment of renal function”, “renal impairment (disorder)”, “renal insufficiency”, “renal impairment” and “renal failure”, as used herein, refers to a medical condition in which the kidneys fail to adequate filter waste products from the blood. Renal failure is mainly determined by a decrease in glomerular filtration rate, the rate which blood is filtered in the glomeruli of the kidney. In renal failure, there may be problems with increased fluid in the body (leading to swelling), increased acid levels, raised levels of potassium, decreased levels of calcium, increased level of phosphate, and in later stages, anemia.

The PPAR agonist for use according to the invention can be used at any stage of ACLF. In a particular embodiment, the subject has ACLF grade 2 or 3.

In another embodiment, the subject has ACLF without kidney failure. In a particular embodiment, the subject has ACLF with kidney failure. In another particular embodiment, the subject has AD or ACLF with a non-kidney organ failure and kidney dysfunction.

In another embodiment, the subject is at risk of ACLF. In yet another embodiment, the subject has at least one ACLF precipitating event. In another embodiment, the precipitating event is selected from alcoholic hepatitis; bacterial, fungal or viral infection; sepsis, poisoning; visceral bleeding and drug-induced liver insufficiency. In another embodiment, the precipitating event is bacterial infection. In yet another particular embodiment, the PPAR agonist is for use in a method for the treatment of sepsis-associated AD or ACLF.

In a further embodiment, the PPAR agonist is for use in a method for treating or preventing hepatic encephalopathy. In a particular embodiment, the PPAR agonist is for use in a method for treating or preventing hepatic encephalopathy in a subject with compensated or decompensated cirrhosis, in particular with decompensated cirrhosis. In another embodiment, the PPAR agonist is for use in a method for the treatment of hepatic encephalopathy in a subject with AD or ACLF.

In the context of the present invention, the PPAR agonist is administered to a subject, in a therapeutically effective amount. A “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

The PPAR agonist can be formulated in a pharmaceutical composition further comprising one or several pharmaceutically acceptable excipients or vehicles (e.g. saline solutions, physiological solutions, isotonic solutions, etc.), compatible with pharmaceutical usage and well-known by one of ordinary skill in the art. These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used.

The PPAR agonist may be administered by different routes and in different forms. For example, it may be administered via a systemic way, per os, parenterally, by inhalation, by nasal spray, by nasal instillation, or by injection, such as intravenously, by intramuscular route, by subcutaneous route, by transdermal route, by topical route, by intra-arterial route, etc. Of course, the route of administration will be adapted to the form of the drug according to procedures well known by those skilled in the art.

In a particular embodiment, the compound is formulated as a tablet. In another particular embodiment, the compound is administered orally.

The frequency and/or dose relative to the administration can be adapted by one of ordinary skill in the art, in function of the patient, the pathology, the form of administration, etc. Typically, the PPAR agonist can be administered at a dose comprised between 0.01 mg/day to 4000 mg/day, such as from 50 mg/day to 2000 mg/day, such as from 100 mg/day to 2000 mg/day; and particularly from 100 mg/day to 1000 mg/day. Administration can be performed daily or even several times per day, if necessary. In one embodiment, the compound is administered at least once a day, such as once a day, twice a day, or three times a day. In a particular embodiment, the PPAR agonist is administered once or twice a day. In particular, oral administration may be performed once a day, during a meal, for example during breakfast, lunch or dinner, by taking a tablet comprising the PPAR agonist.

Suitably, the course of treatment with the PPAR agonist is for at least 1 week, in particular for at least 2, 3, 4, 5, 6, 7, 8, 9, 10, 15, 20 or 24 weeks or more. In a particular embodiment, the course of treatment is for at least 1 month, at least 2 months or at least 3 months. In a particular embodiment, the course of treatment is for at least 1 year, or more depending on the condition of the subject being treated.

In a particular embodiment, the PPAR agonist (“the drug”), is for use as the sole active ingredient for the treatment disclosed herein.

In yet another embodiment, the drug is for use in a combination therapy.

In a particular embodiment, the drug is for use in combination with therapy against a precipitating event.

In a particular embodiment, the precipitating event is a bacterial, fungal or viral infection. Accordingly, the drug can be combined with an antimicrobial or antiviral agent. The most suitable agent will be selected depending on the organism or virus responsible for the infection, as is well known in the art. In a particular embodiment, the precipitating event is hepatitis B virus reactivation. In that case, the drug can be combined with nucleoside or nucleoside analogues. Illustrative antiviral drugs include, without limitation, tenofovir, tenofovir alafenamide and entecavir. In another particular embodiment, the precipitating event is a bacterial infection, and the drug can be combined to an antibiotic. Antibiotics useful in the treatment of bacterial infection are well known in the art. Illustrative antibiotic families include, without limitation, beta-lactam antibiotics (such as penicillins), tetracyclines, cephalosporins, quinolones, lincomycins, macrolides, sulfonamides, glycopeptides, aminoglycosides and carbapenems. In a particular embodiment, the drug can be combined to an antibiotic of the carbapenem family, such as ertapenem.

In another particular embodiment, the precipitating event is acute variceal hemorrhage. Accordingly, the drug can be combined with a vasoconstrictor such as terlipressin, somatostatin, or analogues such as octreotide or vapreotide, in particular octreotide. Such treatment may accompany endoscopic therapy (preferably endoscopic variceal ligation, performed at diagnostic endoscopy less than 12 hours after admission). Short-term antibiotic prophylaxis, such as with ceftriaxone, can also be implemented.

In another particular embodiment, the precipitating event is alcoholic hepatitis. Accordingly, the drug can be combined with prednisolone, which is indicated for patients with severe alcoholic hepatitis.

In another particular embodiment, the drug is for use in combination with a supportive therapy. In a particular embodiment, the supportive therapy is a cardiovascular support. For example, the drug can be combined with a therapy for acute kidney injury, such as withdrawal of diuretics or volume expansion (with intravenous albumin). The drug may also be combined with a vasoconstrictor, such as terlipressin or norepinephrine, in particular if there is no response to volume expansion. In a particular embodiment, the supportive therapy is a treatment of encephalopathy. For example, the drug can be combined with lactulose. Optionally, lactulose therapy can be further completed with the administration of enemas to clear the bowel. In case the subject has severe hepatic encephalopathy refractory to lactulose, albumin dialysis may be used. In yet another particular embodiment, the drug can be combined with rifaximin. In a further embodiment, the drug can be combined with lactitol. In a particular embodiment, the supportive therapy is an extracorporeal liver support. For example, an extracorporeal liver-assist device that incorporates hepatocytes can be used. In another embodiment, plasma exchange can be conducted in addition to the administration of the drug as provided herein. In yet another embodiment, the extracorporeal liver support is albumin exchange or endotoxin removal.

The following examples serve to illustrate the invention and must not be considered as limiting the scope thereof.

### Examples

### Chemistry

Chemical names follow IUPAC nomenclature. Starting materials and solvents were purchased from commercial suppliers (Acros Organic, Sigma Aldrich, Combi-Blocks, Fluorochem, Fluka, Alfa Aesar or Lancaster) and were used as received without further purification. Some starting materials can be readily synthesized by a person skilled in the art. Air and moisture sensitive reactions were carried out under an inert atmosphere of nitrogen, and glassware was oven-dried. No attempts were made to optimize reaction yields. Chemical shifts (6) are reported in ppm (parts per million), by reference to the hydrogenated residues of deuterated solvent as internal standard: 2.50 ppm for DMSO-d6, 7.26 ppm for CDCl3, and 3.31, and 4.78 for Methanol-d4. The spectral splitting patterns are designated as follows: s, singlet; d, doublet; dd, doublet of doublets; ddd, doublet of doublet of doublets; t, triplet; dt, doublet of triplets; q, quartet; m, multiplet; br s, broad singlet.

### Animal Experimentation

Manipulation of animals was conducted carefully in order to reduce stress at the minimum. All the experiments were performed in compliance with the guidelines of French Ministry of Agriculture for experiments with laboratory animals (law 87-848). The study was conducted in compliance with Animal Health Regulation (Council directive No. 2010/63/UE of Sep. 22, 2010 and French decree no. 2013-118 of Feb. 1, 2013 on protection of animals).

### Example 1: Synthesis of Compounds According to the Invention

Compounds of formula (I) can be synthetized following general procedures disclosed in WO2005005369, WO2007147879, WO2007147880, WO2008087366 and WO2008087367.

### Example 1a: sodium 2-{4-[3-methoxy-3-(4-trifluoromethoxy-phenyl)-propyl]-2,6-dimethyl-phenoxy}-2-methyl-propionate

2-{4-[3-Methoxy-3-(4-trifluoromethoxy-phenyl)-propyl]-2,6-dimethyl-phenoxy}-2-methyl-propionic acid (prepared as disclosed in WO2007147880) (500 mg, 0.001135 mol) and sodium hydroxide (45 mg, 0.0011 mol) were mixed in methanol (10 mL, 0.2 mol) and stirred at 40° C. (rotavap) for 10 minutes before methanol was evaporated to dryness to give sodium 2-{4-[3-methoxy-3-(4-trifluoromethoxy-phenyl)-propyl]-2,6-dimethyl-phenoxy}-2-methyl-propionate (515 mg) as a white solid.

mp: 284° C.; 1H NMR (D2O, 300 MHz, δ in ppm): 1.1 (s, 6H); 1.55 (m, 1H); 1.7 (m, 1H); 2.0 (s, 6H); 2.1 (m, 1H); 2.2 (m, 1H); 2.8 (s, 3H); 3.8 (t, 1H); 6.5 (s, 2H); 6.95 (m, 4H)—purity (HPLC): 98.7%.

### Example 1b: L-lysine 2-{4-[3-methoxy-3-(4-trifluoromethoxy-phenyl)-propyl]-2,6-dimethyl-phenoxy}-2-methyl-propionate

2-{4-[3-Methoxy-3-(4-trifluoromethoxy-phenyl)-propyl]-2,6-dimethyl-phenoxy}-2-methyl-propionic acid (prepared as disclosed in WO2007147880) (500 mg, 0.001135 mol) and L-Lysine (160 mg, 0.0011 mol) were mixed in methanol (10 mL, 0.2 mol) and stirred at 40° C. (rotavap) for 10 minutes before methanol was evaporated to dryness to give L-lysine 2-{4-[3-methoxy-3-(4-trifluoromethoxy-phenyl)-propyl]-2,6-dimethyl-phenoxy}-2-methyl-propionate (657 mg) as a white solid.

mp: 176° C.—1H NMR (D2O, 300 MHz, δ in ppm): 1.15 (s, 6H); 1.1-1.2 (m, 1H); 1.3-1.5 (m, 2H); 1.5-1.7 (m, 5H); 1.7-1.85 (m, 3H); 1.9-2 (m, 1H); 2.0 (s, 6H); 2.05-2.15 (m, 1H); 2.15-2.2.3 (m, 1H); 2.8-2.95 (m, 2H); 2.85 (s, 3H); 3.65 (t, 1H); 3.9 (t, 1H); 6.5 (s, 2H); 6.95 (m, 4H)—purity (HPLC): 100%

Other salts of compounds disclosed in WO2007147880 or WO2007147879 can be produced according to the preceding methods.

### Example 1c: 2-(4-(3-methoxy-3-(4-(trifluoromethyl)phenyl)propyl)-2,6-dimethylphenoxy)-2-methylpropanoic acid

2-(4-(3-methoxy-3-(4-(trifluoromethyl)phenyl)propyl)-2,6-dimethylphenoxy)-2-methylpropanoic acid can be prepared as disclosed in WO2007147880.

1H NMR (300 MHz, DMSO d6, δ in ppm): 1.31 (s, 6H); 1.76-2.00 (m, 2H); 2.10 (s, 6H); 2.36-2.58 (m, 2H); 3.14 (s, 3H); 4.24 (m, 1H); 6.77 (s, 2H); 7.51 (d, 2H, J=8.2 Hz); 7.72 (d, 2H, J=8.2 Hz); 12.77 (br s, 1H)—purity (HPLC): 99.7%—mass: 447 MNa+

### Example 1d: 2-[2,6-dimethyl-4-[3-[4-bromophenyl]-3-methoxypropyl]phenoxy]-2-methylpropanoic acid

2-[2,6-dimethyl-4-[3-[4-bromophenyl]-3-methoxypropyl]phenoxy]-2-methylpropanoic acid can be prepared as disclosed in WO2007147880.

1H NMR (300 MHz, DMSO d6, δ in ppm): 1.31 (s, 6H); 1.74-1.98 (m, 2H); 2.10 (s, 6H); 2.33-2.43 (m, 2H); 3.10 (s, 3H); 4.11 (t, 1H, J=5.5 Hz); 6.76 (s, 2H); 7.25 (d, 2H, J=8.5 Hz); 7.55 (d, 2H, J=8.1 Hz); 12.79 (br s, 1H)—purity (HPLC): 99%—mass: 457/459 MNa+

### Example 2: The Compounds According to the Invention Inhibit Monocyte Differentiation into Macrophages

In order to test the efficacy of the compounds to inhibit the activation of the immune system, we used the human monocytic cell line THP-1 (Sigma). THP1 monocytes were cultured in RPMI 1640 with L-glutamine medium (#10-040-CV, Corning) supplemented with 10% fetal bovine serum (FBS, #10270, Gibco), 1% penicillin/streptomycin (#15140, Gibco) and 25 mM Hepes (H0887, Sigma) in a 5% C02 incubator at 37° C.

To evaluate the effect of the compounds on monocyte differentiation, 2.5×10⁴ THP-1 cells were cultured for 24 h in a 384-well plate in FBS-deprived culture medium containing Cpd.1 in dose ranges, as well as 5 or 100 ng/mL phorbol 12-myristate 13-acetate (PMA, #P8139, Sigma), as indicated, to induce differentiation into macrophages.

Tumor necrosis α (TNFα) and monocyte chemoattractant protein 1 (MCP1) were measured in cell supernatants by Homogeneous Time Resolved Fluorescence (HTRF, #62HTNFAPEG for TNFα, and 62HCCL2PEG for MCP1, Cisbio). Fluorescence was measured with Infinite 500 (#30019337, Tecan) to determine the concentration of cytokines.

### Results

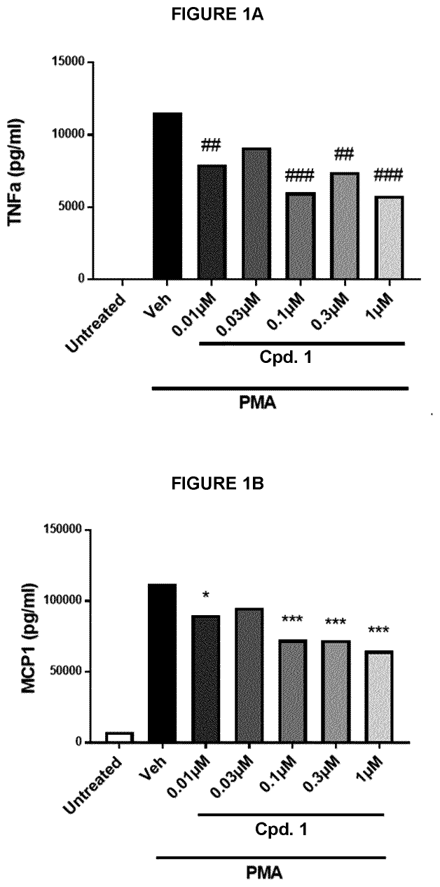

The results are shown in FIGS. 1A and 1B. While PMA induces the secretion of TNFα and MCP1, markers of differentiation of monocytes to macrophages, Cpd.1 drastically reduces TNFα (FIG. 1A) and MCP1 (FIG. 1B) content in the supernatant, reaching circa 50% inhibition at the dose of 1 μM.

Taken together, these results show the efficacy of the compounds according to the invention to reduce the immune system activation.

### Example 3: The Compounds According to the Invention Inhibit Macrophage Activation

In order to test the efficacy of the compounds on macrophage activation and pro-inflammatory cytokine production, 2.5×10⁴ THP-1 cells were cultured in a 384-well plate and treated with 100 ng/mL PMA (#P8139, Sigma) for 24 h to induce differentiation into macrophages.

Then, medium was removed and FBS-deprived medium containing the compound of formula (I) was added for 24 h. Finally, THP1 macrophages were stimulated with 100 ng/mL LPS (E. coli 055:B5, #L4005, Sigma) for 6 h.

### Results

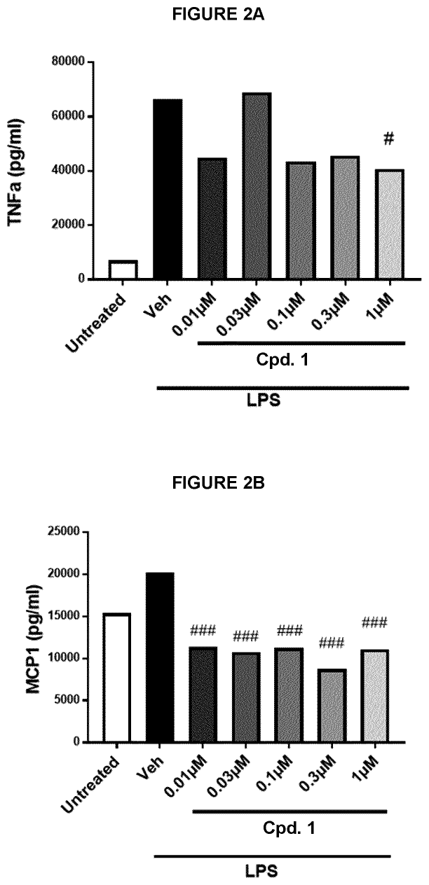

The results are shown in FIG. 2. Cpd.1 was potent at reducing TNFα and MCP1 secretion (FIGS. 2A and 2B), overpassing the untreated condition for MCP1 secretion at all doses tested (FIG. 2B).

These results show the potency of the compounds of formula (I) to counteract macrophage activation and limit cytokines production, thereby protecting damages to the tissues.

### Example 4: Compounds According to the Invention Reduce Circulating Cytokine Levels in a Model of Endotoxemia

Increased circulating endotoxins, such as lipopolysaccharide (LPS), is associated with the risk of developing ACLF (Takaya, J Clin Med. 2020). Cirrhotic patients often present with altered intestinal barrier, which increases bacterial translocation and inflammation. The activation of immune cells, such as macrophages, by LPS produces inflammatory cytokines that induce parenchymal cell death in different tissues (liver, kidney, etc) which can eventually lead to multiple organ failure.

### Preclinical Model of Endotoxemia

To evaluate the efficacy of the compounds to reduce the cytokine production in response to LPS-induced endotoxemia, male Sprague Dawley rats of 250-275 g from Janvier Labs (France) received a single intraperitoneal injection of 1 mg/kg LPS (Escherichia coli 0111:B4, #L2630, Sigma).

Cpd.1 (3 mg/kg/day) or vehicle (Labrafil M 1944 CS, #3063, Gattefossé) was administered by oral gavage during the 3 days before LPS injection. Rats were euthanized by cervical dislocation 3 hours after treatment. Blood samples were obtained from retro-orbital sinus puncture on animals slightly asleep with isoflurane (Isoflurin 1000 mg/g, GTIN 03760087152678, Axience) just before sacrifice.

The serum concentrations of interleukin-6 (IL6) and interleukin-1β (L1β) were determined by ELISA (SR6000B and SRLB00, respectively, R&D Systems).

### Results

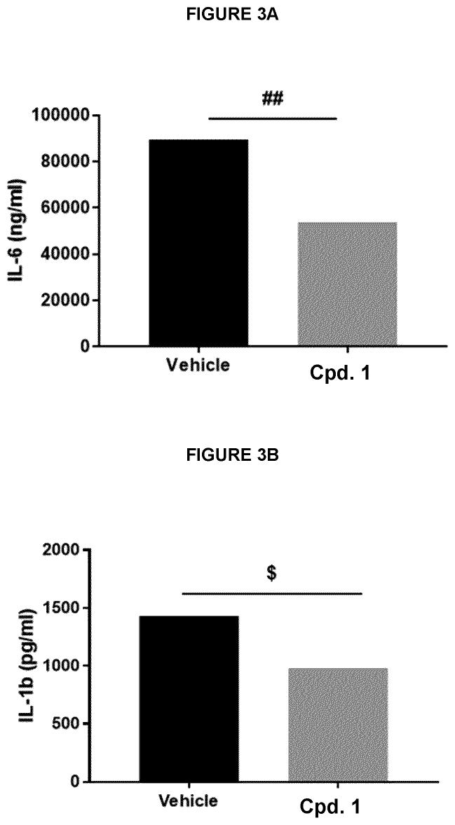

LPS injection induced a high production of the pro-inflammatory cytokines IL6 and L1β, reaching 80 μg/mL and 1500 μg/mL, respectively, in the sera (undetectable in healthy animals). Cpd.1 drastically reduced IL6 and IL1β levels by 40% and 31%, respectively (FIGS. 3A and 3B).

These results show that treatment with Cpd.1 reduces circulating cytokines levels in response to LPS in vivo, thereby protecting from tissue damages and organ failure.

### Example 5: Compounds According to the Invention Improve Hepatic Function and Cytokine Level in a Model of Endotoxemia

Increased endotoxins, such as lipopolysaccharide (LPS), is associated with the risk of developing ACLF (Takaya, J Clin Med. 2020, 9(5), p 1467). In patients with such severe liver diseases, serum albumin levels are low due to a reduction of the hepatocyte mass whereas total bilirubin levels are increased. These indicators are therefore useful markers to assess the effect of molecules on liver function upon acute liver injury.

### Preclinical Model of Endotoxemia

To evaluate the efficacy of the compounds on hepatic markers concentration in response to LPS-induced endotoxemia, male Sprague Dawley rats of 250-275 g from Janvier Labs received a single intraperitoneal injection of 1 mg/kg LPS (Escherichia coli 0111:B4, #L2630, Sigma-Aldrich).

Cpd.1 (3 mg/kg/day), Cpd.19 (100 mg/kg/day) or vehicle (Labrafil M 1944 CS, #3063, Gattefossé for Cpd.1 or carboxymethylcellulose 1%, 0.1% Tween 80 for Cpd.19) was administered by oral gavage during the 3 days before LPS injection. Rats were euthanized by cervical dislocation 3 hours after LPS treatment. Blood samples were obtained from retro-orbital sinus puncture on animals slightly asleep with isoflurane (Isoflurin 1000 mg/g, GTIN 03760087152678, Axience) just before sacrifice.

### Evaluation of Hepatic Markers Concentration in Rat Serum

The serum concentration of total bilirubin was measured using the Randox kit for Daytona plus automate (#BR3859, Randox Laboratories). Briefly, total bilirubin is quantified by a colorimetric assay based on the method described by Jendrassik L, and Gróf P., Biochem Zeitschrift 1938, 297, p 82-9.

The serum concentration of albumin was measured using the Randox kit for Daytona plus automate (#AB8301, Randox Laboratories). Briefly, the measurement of albumin is based on its quantitative binding to the indicator 3,3′,5,5′-tetrabromo-m cresol sulphonphthalein (bromocresol green). The albumin-BCG-complex absorbs maximally at 578 nm.

### Cytokine Analysis in Rat Serum

The concentration of tumor necrosis α (TNFα) was determined using a multiplex sandwich ELISA system (Rat Premixed Multi-Analyte Kit LXSARM, Biotechne) according to the manufacturer instructions. Briefly, serum samples were added onto magnetic particles pre-coated with cytokines-specific antibodies. After washing, cytokines were detected through the addition of biotinylated antibodies. Finally, streptavidin conjugated with phycoerythrin were added and analysis were carried out with the Luminex 200 analyzer. The signal strength of phycoerythrin is proportional to the concentration of the specific cytokine.

### Results

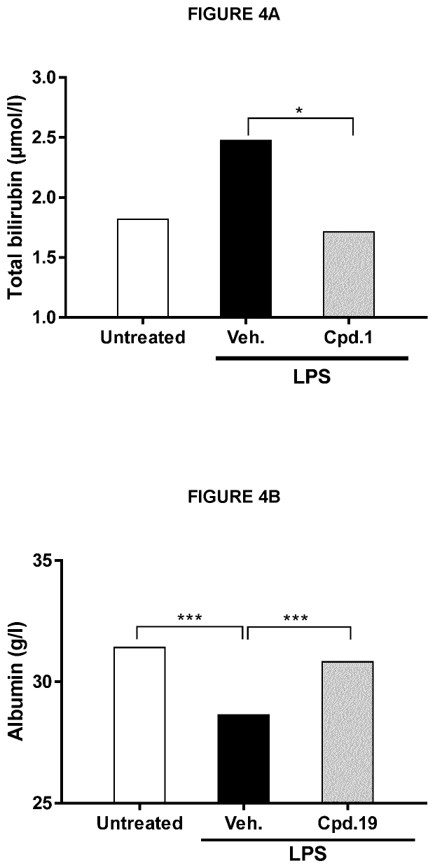

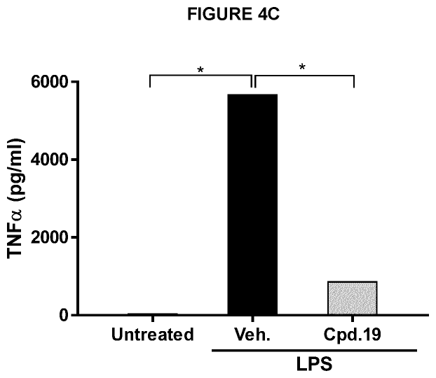

Rats undergoing endotoxemia had altered hepatic function as shown by high total bilirubin concentration in the serum (FIG. 4A). When administrated to rats undergoing endotoxemia, Cpd.1 completely restored the level of total bilirubin, compared to the vehicle condition.

In parallel, while LPS injection led to a decrease of the serum albumin level, animals treated with Cpd.19 showed a restoration of albumin concentration by 79% when compared to the vehicle control rats (FIG. 4B).

Regarding circulating cytokines, we demonstrated that Cpd.19 drastically reduced the LPS-induced serum TNFα level by 85% (FIG. 4C).

These results show that administration of both Cpd. 1 and Cpd.19 allows to improve hepatic function and inflammation in response to LPS in vivo, thereby protecting from tissue damages and organ failure.

### Example 6: Compounds According to the Invention Reduce Hepatic and Systemic Inflammation and Improve Hepatic Function in a Model of Acute Liver Failure

Low doses of LPS in combination with the hepatotoxic agent D-Galactososamine (GaIN) promote specific liver injury in mice and induce the inflammatory cytokines production, thus recapitulating the clinical picture of acute liver injury in human (Pourcet et al., Gastroenterology, 2018, 154(5), p 1449-1464.e20). Hepatic injury induced by LPS/GaIN is therefore a widely used mouse model to understand the effect of pharmacological agents on fulminant hepatitis.

### Preclinical Model of Acute Liver Failure

To evaluate the efficacy of the compounds on the inflammatory response occurring upon acute liver failure, C57BL/6J male mice (8 weeks old, Janvier Labs) received an intraperitoneal injection of 0.025 mg/kg LPS (Escherichia coli 0111:B4, #L2630, Sigma-Aldrich) supplemented with 700 mg/kg D-Galactosamine (GaIN, G0500, Sigma-Aldrich).

Cpd.1 (3 mg/kg/day), Cpd.18 (1 mg/kg/day) or vehicle (carboxymethylcellulose 1%, 0.1% Tween 80) was administered by oral gavage during the three days before LPS/Gal-N injection. Mice were sacrificed 4 h after LPS/GaIN injection and liver tissues were subsequently collected. Blood samples were obtained from retro-orbital sinus puncture on animals slightly asleep with isoflurane (Isoflurin 1000 mg/g, GTIN 03760087152678, Axience) just before sacrifice.

### Hepatic Gene Expression Analysis

Total RNA were isolated from mouse liver using the NucleoSpin 8 RNA Core kit (Macherey Nagel) following manufacturer's instructions. Reverse transcription was performed using M-MLV RT (Moloney Murine Leukemia Virus Reverse Transcriptase) (#28025, Invitrogen) in 1×RT buffer, 0.5 mM DTT, 0.18 mM dNTPs, 200 ng random primers and 30 U RNase inhibitor.

Quantitative PCR (RT-qPCR) was then carried out using the CFX96 Touch™ Real-Time PCR Detection System (Biorad). Briefly, the PCR reactions were performed in 10 μl reaction containing iQ SYBR Green Supermix (BioRad) and 0.25 mM of each primer. Each nucleotide sequences used in this study are described below:

| Gene | NCBI | | |
|---|---|---|---|
| name | reference | Forward sequence | Reverse sequence |
| Rplp0 | NM_007475.5 | CATGCTCAACATCTCCCCCTT | GGGAAGGTGTAATCCGTCTCCA |
| (36B4) | | CTCC | CAG |
| Ccl2 | NM_011333 | GCTACAAGAGGATCACCAGCA | GTCTGGACCCATTCCTTCTTGG |
| (Mcp1) | | G | |
| IL6 | NM_031168 | CAGAGTCCTTCAGAGAGATAC | ATGGTCTTGGTCCTTAGCC |
| | | AG | |
| TNF | NM_013693 | CGTGGAACTGGCAGAAGAGG | AGACAGAAGAGCGTGGTGGC |
| (TNFα) | | | |
| IL1b | NM_008361 | AGACTTCCATCCAGTTGCCTT | TTGTTGATGTGCTGCTGTGA |
| | | CTTG | |
| Cd68 | NM_001291058.1 | CTACATGGCGGTGGAGTACAA | GAAACTGCCATGTTTGAGCA |

The specificity of the amplification was checked by recording the dissociation curves and the efficiency was verified to be above 95% for each primer pairs. mRNA levels were normalized to the expression of Rplp0 housekeeping gene and the fold induction was calculated using the cycle threshold (ΔΔCT) method.

### Cytokines Analyses in Mice Serum

The concentrations of interleukin-6 (IL6), tumor necrosis α (TNFα), CC-Motif Chemokine Ligand 2 (CCL2, MCP1) and interleukin-10 (IL10), and were determined using a multiplex sandwich ELISA system (Mouse Magnetic Luminex #LSXAMSM-06, Biotechne) according to the manufacturer instructions. Briefly, serum samples were added onto magnetic particles pre-coated with cytokines-specific antibodies. After washing, cytokines were detected through the addition of biotinylated antibodies. Finally, streptavidin conjugated with phycoerythrin were added and analysis were carried out with the Luminex 200 analyzer. The signal strength of phycoerythrin is proportional to the concentration of the specific cytokine.

### Evaluation of Albumin Concentration in Mice Serum

The serum concentration of albumin was measured using the Randox kit for Daytona plus automate (#AB8301, Randox Laboratories). Briefly, the measurement of albumin is based on its quantitative binding to the indicator 3,3′,5,5′-tetrabromo-m-cresol-sulphonaphthalein (bromocresol green). The albumin-BCG-complex absorbs maximally at 578 nm.

### Results

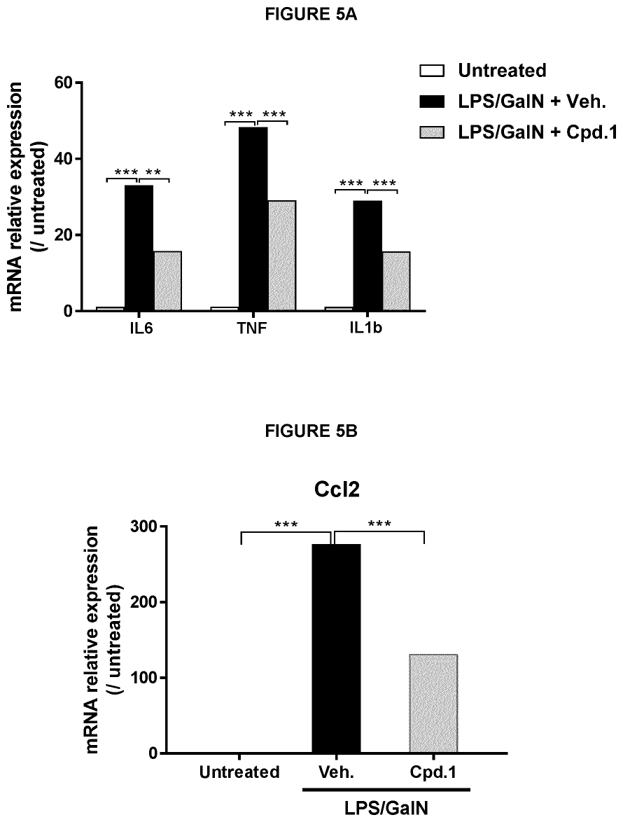

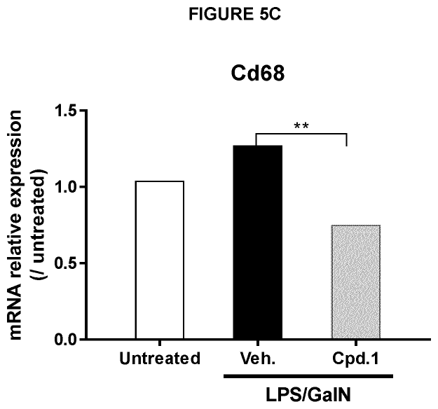

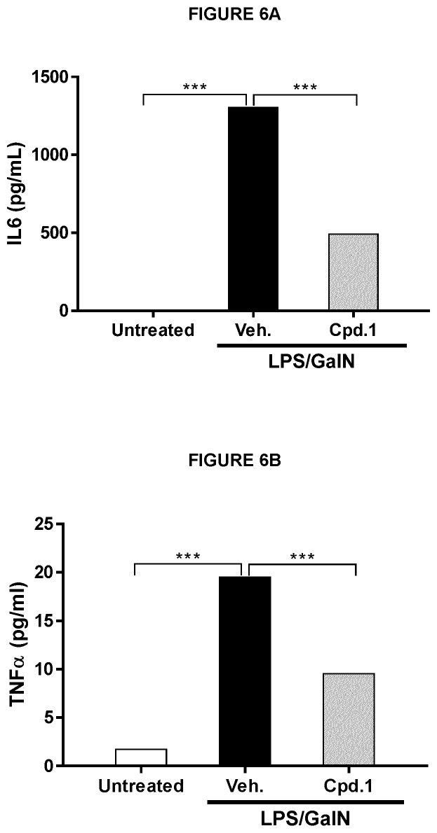

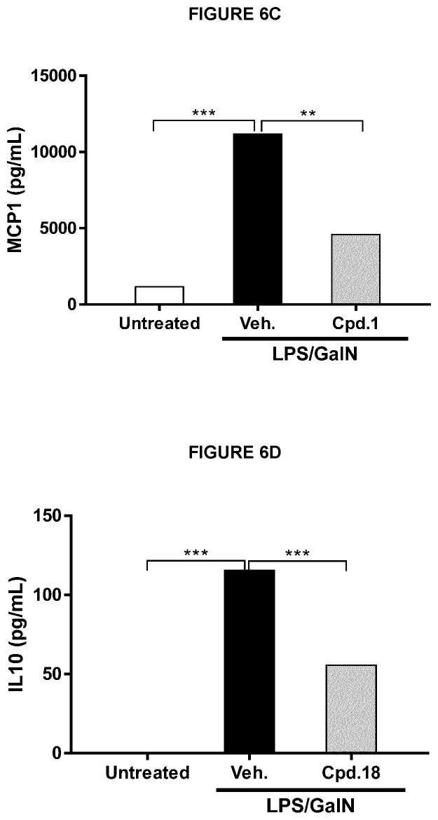

Mice injected with LPS/GaIN showed a strong increase in hepatic mRNA expression of genes encoding Interleukin-6 (IL6), Tumor necrosis factor (TNF), interleukin-1β (IL1b) and CC-Motif Chemokine Ligand 2 (Ccl2, Mcp1) (FIG. 5A-B) by 25 to 250-fold compared to the untreated mice. The induction of proinflammatory cytokines expression level thereby validated the present model of acute liver failure in mice. Interestingly, Cpd.1 administration was accompanied by a decrease of LPS/GaIN-induced cytokines expression i.e., IL6, TNF, IL1b and Ccl2 by 52%, 40%, 46% and 52% respectively (FIG. 5A-B). Cpd.1 also reduced the mRNA expression level of the macrophage marker Cd68 by 41% (FIG. 5C), indicating a modification in the level of immune cells recruitment within the liver. Consistently, the circulating cytokine levels of IL6, TNFα and CCL2 were also reduced upon Cpd.1 treatment by 62%, 51% and 59%, respectively; compared to the vehicle condition (FIG. 6A-C).

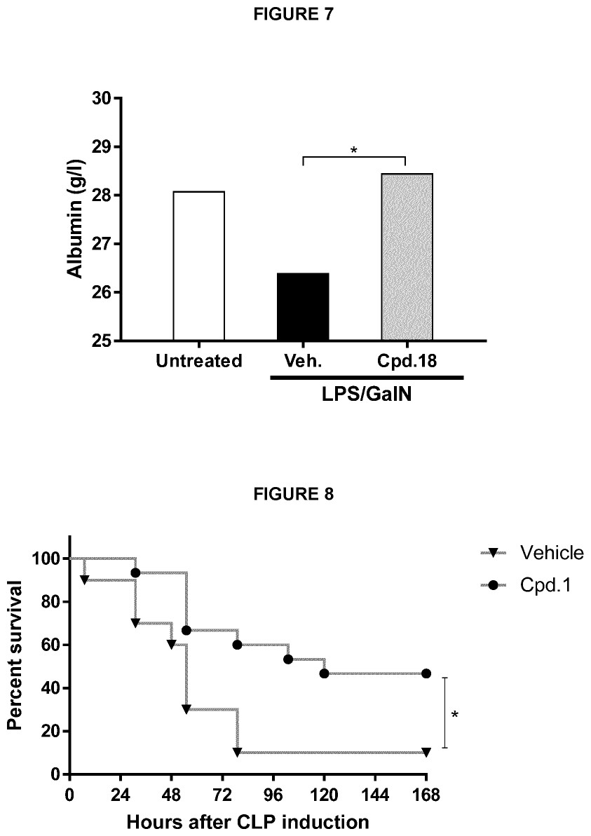

This model was also used to assess the effect of Cpd.18 on acute liver injury markers. The level of circulating IL10 induced by LPS/GaIN was decreased by 52% in comparison with the vehicle condition (FIG. 6D). In parallel, while LPS/GaIN injection in mice led to a decrease of the serum albumin level, Cpd.18 treatment allowed to restore albumin concentration (FIG. 7).

Altogether, these results demonstrate that Cpd.1 and Cpd.18 reduce hepatic and systemic inflammation and improve hepatic function in mice undergoing acute liver failure.

### Example 7: Compounds According to the Invention Improves Survival in a Model of Sepsis

### Aim of the Study

ACLF is a rare clinical condition but remains associated with high short-term mortality either during hospitalization stay or shortly after discharge. A consensual paradigm is emerging implying an overactivation of the innate immune system due to translocation of bacterial products like PAMPs (mainly LPS from Gram negative bacteria) with or without living bacteria from the gut. Such an impaired intestinal barrier provokes an exaggerated endotoxemia resulting in an uncontrolled inflammatory storm which can jeopardize minimal functioning of cirrhotic liver and other vital organs like the kidneys, the brain, the coagulation system, the cardiovascular system and/or the respiratory system.

Polymicrobial sepsis induced by cecal ligation and puncture (CLP) is characterized by dysregulated systemic inflammatory responses followed by immunosuppression. The CLP model in mice mimics the progression and features of human sepsis and is thus also useful to determine whether a drug would be useful in the treatment of ACLF in view of the common pathophysiological features of transition from decompensated cirrhosis to ACLF and from sepsis to septic shock.

This study aims to investigate the efficacy of Cpd.1 in cecal ligation and puncture (CLP) model in C57BL6J (BL6) male mice. The efficacy of the test compound was evaluated based on the survival rate of the animals within the study period.

### Cecal Ligature and Puncture Surgery

C57BL6J male mice (supplier Janvier—France) at 9 weeks of age and weighing 23-25 g on arrival were anesthetized with 250 μL of xylazine/ketamine solution (20 mg/100 g body weight) by intraperitoneal route. A 1-1.5 cm abdominal midline incision was made, and the caecum was located and tightly ligated at half the distance between distal pole and the base of the cecum with 4-0 silk suture (mild grade). The caecum was punctured through-and-through once with a 21-gauge needle from mesenteric toward antimesenteric direction after medium ligation. A small amount of stool was extruded to ensure that the wounds were patent. Then the cecum was replaced in its original position within the abdomen, which was closed with sutures and wound clips. Mice were followed for body weight evolution and mortality rate until Day 6.

### Treatment Scheme

Cpd.1 or vehicle (Labrafil M 1944 CS, #3063, Gattefossé) was administered at 0.3 mg/kg, p.o. for three days before CLP surgery. The day of CLP (day 0), Cpd.1 was administrated 1 h before surgery and pursued daily until day 6. Experiment was terminated at day 7.

### Experimental Groups

Two groups of C57BL6J mice at the age of 9 weeks were treated as described above.
1. BL6 mice CLP (21 G needle)+Vehicle (0.3 mL/kg; p.o.) (10 mice)
2. BL6 mice CLP (21 G needle)+Cpd.1 (0.3 mg/kg; p.o.) (15 mice)

### Clinical Signs

No side effect of Cpd.1 was recorded after each administration. Body weight and survival rate were measured for 7 days.

### Survival Rates Results

The “CLP+Vehicle (p.o.)” group reached 30% of survival rate at Day 2 and 10% at Day 7, until the end of the experiment.
The “CLP+Cpd.1 (0.3 mg/kg, p.o.)” group showed a significant increase in survival rate by 47% at the end of the experiment (Day 7) compared to the “CLP+Vehicle (p.o.)” control group (FIG. 8).

### Conclusion

Cpd.1 (0.3 mg/kg, p.o.) given 3 days before surgery, 1 h before surgery and once daily until Day 7, significantly improved the survival rate in comparison with CLP+vehicle control group. In conclusion, Cpd.1 has a beneficial effect on survival rate in CLP induced polymicrobial sepsis in mice.

### Example 8: Compounds According to the Invention Inhibit Macrophage Activation

In order to test the efficacy of the compounds on macrophage activation and pro-inflammatory cytokine production, 2.5×10⁴ THP-1 cells were cultured in a 384-well plate and treated with 100 ng/mL PMA (#P8139, Sigma) for 24 h to induce differentiation into macrophages.

Then, medium was removed and FBS-deprived medium containing 1 or 10 μM compound was added for 24 h. Finally, THP1 macrophages were stimulated for 6 h with 100 ng/mL LPS (Klebsiella pneumoniae, #L4268, Sigma-Aldrich).

Monocyte chemoattractant protein 1 (MCP1) and Tumor necrosis α (TNFα) were measured in cell supernatants by Homogeneous Time Resolved Fluorescence (HTRF, #62HTNFAPEG for TNFα, and 62HCCL2PEG for MCP1, Cisbio). Fluorescence was measured with Infinite 500 (#30019337, Tecan) to determine the concentration of cytokines.

### Results

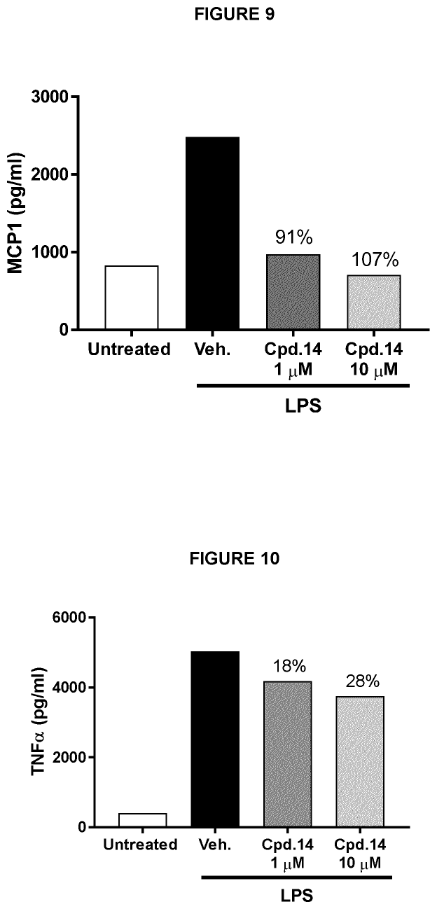

Treatment of macrophages with LPS from Klebsiella led to a 3,5-fold and 13-fold increase of MCP1 and TNFα serum levels, respectively (FIGS. 9 and 10). In order to evaluate the effect of the compounds according to the invention on LPS-induced cytokines production by macrophages, the percentage of inhibition over the vehicle condition were calculated for each compound at 1 and 10 μM. The results are presented in Table 1 for MCP1 and Table 2 for TNFα. The results obtained for Cpd.14 are shown through graphs as representative experiments (FIGS. 9 and 10).

TABLE 1

% inhibition of MCP1
secretion over mean Veh.

| Compound | 1 μM | 10 μM |
|---|---|---|
| Cpd. 1 | 108 | 120 |
| Cpd. 2 | 96 | 106 |
| Cpd. 3 | 90 | 107 |
| Cpd. 4 | 100 | 110 |
| Cpd. 5 | 93 | 110 |
| Cpd. 6 | 94 | 103 |
| Cpd. 7 | 95 | 107 |
| Cpd. 9 | 86 | 127 |
| Cpd. 13 | 99 | 111 |
| Cpd. 14 | 91 | 107 |
| Cpd. 17 | 22 | 54 |
| Cpd. 18 | 93 | 123 |
| Cpd. 19 | 24 | 89 |
| Cpd. 20 | 105 | 120 |
| Cpd. 21 | 80 | 121 |
| Cpd. 22 | 79 | 117 |
| Cpd. 23 | 100 | 126 |

TABLE 2

% inhibition of TNFα
secretion over mean Veh.

| Compound | 1 μM | 10 μM |
|---|---|---|
| Cpd. 3 | 0 | 20 |
| Cpd. 7 | 25 | 42 |
| Cpd. 9 | 3 | 77 |
| Cpd. 13 | 0 | 18 |
| Cpd. 14 | 18 | 28 |
| Cpd. 21 | 0 | 19 |
| Cpd. 22 | 36 | 11 |
| Cpd. 23 | 15 | 32 |

As shown in example 3, Cpd.1 reduced the production of MCP1 induced by LPS Klebsiella, overpassing the untreated condition for MCP1 secretion (FIG. 9B). Likewise, treatment with 10 μM of Cpd.2, Cpd.3, Cpd.4, Cpd.5, Cpd.6, Cpd.7, Cpd.9, Cpd.13, Cpd.14, Cpd.17, Cpd.18, Cpd.19, Cpd. 20, Cpd.21, Cpd.22, and Cpd.23 decreased MCP1 secretion from 54 to 132% (Table 1).

In parallel, when added at 10 μM, Cpd.3, Cpd.7, Cpd.9, Cpd.13, Cpd.14, Cpd.21, Cpd.22 and Cpd.23 also reduced TNFα secretion by macrophages from 11 to 58% (Table 2).

These results show the potency of the compounds to counteract macrophage activation and limit cytokines production, thereby protecting damages to the tissues.

### Example 9: Compounds According to the Invention Protect Hepatocyte from Apoptosis

In order to evaluate the effect of the compounds on human hepatocytes that undergo a cellular stress induced by staurosporin, the human hepatoblastoma-derived HepG₂ cell line (ECACC, #85011430, Sigma-Aldrich) was cultured in high-glucose DMEM medium (#41965, Gibco, France) supplemented with 10% of fetal bovine serum (FBS, #10270, Gibco), 1% penicillin/streptomycin (#15140, Gibco), 1% sodium pyruvate (#11360, Gibco) and 1% MEM non-essential amino acids (#11140, Gibco) in a 5% CO₂ incubator at 37° C.

To evaluate caspase 3/7 activity, which is a surrogate marker of apoptosis, 1.5×10⁴ cells were plated in a 384-well plate (#781080, Greiner, France). After cell adherence (8 hours), cells were serum starved for 16 h in the presence of 0.3 μM of compounds or vehicle. Cpd.1 was also used at 3 and 10 μM. Thereafter, cells were treated with 10 μM staurosporin (#569397, Sigma-Aldrich, Germany) supplemented with compound for additional 4 hours before cell lysis and caspase activity measurement.

Caspase 3/7 activity was measured using Caspase Glow™ 3/7 assay (#G8093, Promega, USA). Luminescence was measured using a Spark microplate reader (#30086376, Tecan, USA). The amount of luminescence (RLU) directly correlates with caspase 3/7 activity.

### Results

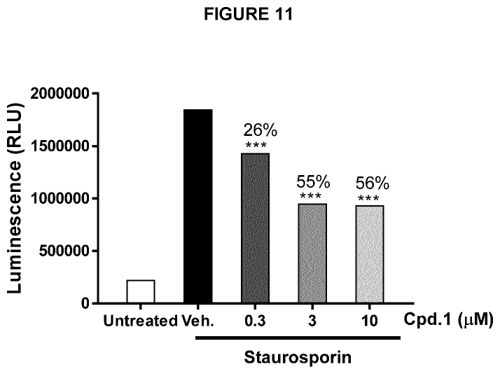

Incubation of HepG2 cells with staurosporin induced apoptosis, as shown by an increase of caspase 3/7 activity by 8-fold (FIG. 11). In order to evaluate the effect of the compounds according to the invention on staurosporin-induced caspase activity in HepG2 cells, the percentage of inhibition over the vehicle condition were calculated. Interestingly, the addition of Cpd.1 reduced caspase activity induced by staurosporin in a dose-dependent manner (FIG. 11). Likewise, treatment with 0.3 μM of Cpd.2, Cpd.4, Cpd.6, Cpd.7, Cpd.13, Cpd.14 or Cpd.21 also decreased staurosporin-induced caspase activity from 26 to 79% (Table 3). These results show that the compounds directly protect hepatocyte from cell death by inhibiting caspase activity.

TABLE 3

| | % Inhibition of caspase |
|---|---|
| | activity over mean Veh. |
| Compound | 0.3 μM |
| Cpd. 1 | 26 |
| Cpd. 2 | 70 |
| Cpd. 4 | 32 |
| Cpd. 6 | 43 |
| Cpd. 7 | 66 |
| Cpd. 13 | 79 |
| Cpd. 14 | 77 |
| Cpd. 21 | 60 |

Altogether, these results show that treatment with compounds according to the invention reduce overt activation of the immune system in endotoxemia and sepsis, via direct anti-inflammatory effects on monocytes and macrophages in one hand, while they also directly reduce apoptosis on the other. Therefore, the compounds according to the invention protect from tissue damages, organ failure and death that occur in ACLF.

1. A method for the treatment of liver failure in a subject in need thereof, comprising the step of administering a PPAR agonist selected from:
lanifibranor, bezafibrate, fenofibrate, pemafibrate, seladelpar, saroglitazar, pioglitazone and rosiglitazone; or
a compound of formula (I), or a pharmaceutically acceptable salt thereof, 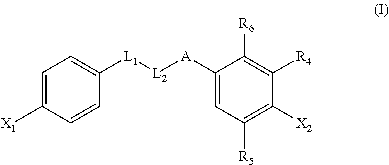
wherein:
X1 represents a halogen atom, a R1 group or a G1-R1 group;
L1 represents a bond, a thiophenyl group or a thiazole group substituted or not by a (C1-C3)alkyl group;
L2 represents:
(i) a —CH—OR7 group, in which R7 represents a hydrogen atom, an unsubstituted (C1-C6)alkyl group or a (C1-C6)alkyl group substituted by a (C6-C14)aryl group;
(ii) a carbonyl group (CO); or
(iii) a C═N—OR8, in which R8 represents an unsubstituted (C1-C6)alkyl group;
A represents a CH═CH or a CH₂—CH₂ group;
X2 represents a G2-R2 group;
G1 and G2, identical or different, represent an atom of oxygen or sulfur;
R1 represents a hydrogen atom, an unsubstituted (C1-C6)alkyl group, a (C6-C14)aryl group or an alkyl group that is substituted by at least one substituent selected from halogen atoms, (C1-C6)alkoxy groups, (C1-C6)alkylthio groups, (C5-C10)cycloalkyl groups, (C5-C10)cycloalkylthio groups and 5- to 14-membered heterocyclic groups;
R2 represents a (C1-C6)alkyl group substituted by a —COOR3 group;
R3 represents a hydrogen atom or a (C1-C6)alkyl group that is substituted or not by at least one substituent selected from halogen atoms, (C5-C10)cycloalkyl groups and 5- to 14-membered heterocyclic groups;
R4 represents a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups;
R5 represents a hydrogen atom, a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups; and
R6 represents a hydrogen atom or a halogen atom;
with the proviso that said compound of formula (I) is not:
elafibranor or a pharmaceutically acceptable salt thereof; or
2-[2,6-dimethyl-4-[3-[4-(methylthio)phenyl]-3-oxo-propyl]phenoxy]-2-methylpropanoic acid or a pharmaceutically acceptable salt thereof.

2. The method according to claim 1, wherein said PPAR agonist is a compound of formula (I) wherein X1 is a G1-R1 group and G1 is an oxygen atom.

3. The method according to claim 1, wherein said PPAR agonist is a compound of formula (I) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group; and
L2 is a —CH—OR7 group.

4. The method according to claim 1, wherein said compound is selected from the group consisting of:
2-[4-(3-methoxy-3-(4-(trifluoromethoxy)phenyl)propyl)-2,6-dimethylphenoxy]-2-methylpropanoic acid or a pharmaceutically acceptable salt thereof;
2-[2,6-dimethyl-4-[3-[4-(trifluoromethyloxy)phenyl]-3-oxo-propyl]phenoxy]-2-methylpropanoic acid or a pharmaceutically acceptable salt thereof;
2-[2,6-dimethyl-4-[3-[4-(trifluoromethyl)phenyl]-3-oxo-propyl]phenoxy]-2-methylpropanoic acid or a pharmaceutically acceptable salt thereof;
2-[2,6-dimethyl-4-[3-[4-(methylthio)phenyl]-3-methoxypropyl]phenoxy]-2-methylpropanoic acid or a pharmaceutically acceptable salt thereof;
2-[2,6-dimethyl-4-[3-[4-(methylthio)phenyl]-3-isopropyloxypropyl]phenoxy]-2-methylpropanoic acid or a pharmaceutically acceptable salt thereof;
2-(4-(3-hydroxy-3-(4-(trifluoromethoxy)phenyl)propyl)-2,6-dimethylphenoxy)-2-methylpropanoic acid or a pharmaceutically acceptable salt thereof;
2-(4-(3-(methoxyimino)-3-(4-(trifluoromethoxy)phenyl)propyl)-2,6-dimethylphenoxy)-2-methylpropanoic acid or a pharmaceutically acceptable salt thereof;
2-(2-chloro-4-(3-(4-methyl-2-(4-(trifluoromethyl)phenyl)-thiazol-5-yl)-3-oxopropyl)phenoxy)-2-methylpropanoic acid or a pharmaceutically acceptable salt thereof;
2-(2,3-dichloro-4-(3-ethoxy-3-(4-methyl-2-(4-(trifluoromethyl)-phenyl)thiazol-5-yl)propyl)phenoxy)-2-methylpropanoic acid or a pharmaceutically acceptable salt thereof;
2-(4-(3-(benzyloxy)-3-(5-(4-(trifluoromethyl)phenyl)thien-2-yl)propyl)-2,3-dichlorophenoxy)-2-methylpropanoic acid or a pharmaceutically acceptable salt thereof;
2-(2,3-dichloro-4-(3-methoxy-3-(5-(4-(trifluoromethyl)phenyl)-thien-2-yl)propyl)phenoxy)-2-methylpropanoic acid or a pharmaceutically acceptable salt thereof;
2-(4-(3-methoxy-3-(4-(trifluoromethyl)phenyl)propyl)-2,6-dimethylphenoxy)-2-methylpropanoic acid or a pharmaceutically acceptable salt thereof; and
2-[2,6-dimethyl-4-[3-[4-bromophenyl]-3-methoxypropyl]phenoxy]-2-methylpropanoic acid or a pharmaceutically acceptable salt thereof.

5. The method according to claim 4, wherein said compound is 2-[4-(3-methoxy-3-(4-(trifluoromethoxy)phenyl)propyl)-2,6-dimethylphenoxy]-2-methylpropanoic acid or a pharmaceutically acceptable salt thereof.

6. The method according to claim 1, wherein the liver failure is selected from acute decompensation (AD), acute on chronic liver failure (ACLF), acute liver failure (ALF) and decompensated cirrhosis.

7. The method according to claim 1, wherein the subject has AD, decompensated cirrhosis with or without ACLF, or is at risk of AD and ACLF.

8. The method according to claim 1, wherein the subject has decompensated cirrhosis or is at risk of decompensated cirrhosis or acute decompensation.

9. The method according to claim 1, wherein the subject has decompensated cirrhosis.

10. The method according to claim 1, wherein the method reverts decompensated cirrhosis to compensated cirrhosis.

11. The method according to claim 1, wherein the method prevents liver decompensation in a subject having ACLF.

12. The method according to claim 1, wherein the liver failure is ALF.

13. The method according to claim 1, wherein the method further prevents kidney failure or hepatic encephalopathy.

14. The method according to claim 1, wherein the subject has ACLF without kidney failure, or wherein the subject has ACLF with a non-kidney organ failure with kidney dysfunction.

15. The method according to claim 1, wherein the liver failure is sepsis-associated ACLF.

16. The method of claim 4, wherein the liver failure is selected from acute decompensation (AD), acute on chronic liver failure (ACLF), acute liver failure (ALF), and decompensated cirrhosis.

17. The method of claim 4, wherein the subject has decompensated cirrhosis or is at risk of decompensated cirrhosis or acute decompensation.

18. The method of claim 4, wherein the liver failure is ALF.

19. The method of claim 5, wherein the liver failure is selected from acute decompensation (AD), acute on chronic liver failure (ACLF), acute liver failure (ALF), and decompensated cirrhosis.

20. The method of claim 5, wherein the subject has decompensated cirrhosis or is at risk of decompensated cirrhosis or acute decompensation.


SEQUENCE LISTING (12 sequences)

SEQ ID NO: 1 - DNA, 25 bp, artificial
  primer
catgctcaacatctcccccttctcc

SEQ ID NO: 2 - DNA, 25 bp, artificial
  primer
gggaaggtgtaatccgtctccacag

SEQ ID NO: 3 - DNA, 22 bp, artificial
  primer
gctacaagaggatcaccagcag

SEQ ID NO: 4 - DNA, 22 bp, artificial
  primer
gtctggacccattccttcttgg

SEQ ID NO: 5 - DNA, 23 bp, artificial
  primer
cagagtccttcagagagatacag

SEQ ID NO: 6 - DNA, 19 bp, artificial
  primer
atggtcttggtccttagcc

SEQ ID NO: 7 - DNA, 20 bp, artificial
  primer
cgtggaactggcagaagagg

SEQ ID NO: 8 - DNA, 20 bp, artificial
  primer
agacagaagagcgtggtggc

SEQ ID NO: 9 - DNA, 25 bp, artificial
  primer
agacttccatccagttgccttcttg

SEQ ID NO: 10 - DNA, 20 bp, artificial
  primer
ttgttgatgtgctgctgtga

SEQ ID NO: 11 - DNA, 21 bp, artificial
  primer
ctacatggcggtggagtacaa

SEQ ID NO: 12 - DNA, 20 bp, artificial
  primer
gaaactgccatgtttgagca